SEQUENCE LISTING (41 sequences)

SEQ ID NO: 1 - protein, 5 aa, Homo sapiens
DYGMH

SEQ ID NO: 2 - protein, 5 aa, Homo sapiens
NYGMH

SEQ ID NO: 3 - protein, 5 aa, Homo sapiens
TYTMH

SEQ ID NO: 4 - protein, 17 aa, Homo sapiens
VIWYDGNNKYYADSVKG

SEQ ID NO: 5 - protein, 17 aa, Homo sapiens
GIWYDGSNKYYADSVKG

SEQ ID NO: 6 - protein, 16 aa, Homo sapiens
AIGTGGGTDYADSVKG

SEQ ID NO: 7 - protein, 8 aa, Homo sapiens
DWGRAFDI

SEQ ID NO: 8 - protein, 7 aa, Homo sapiens
ESPNFDY

SEQ ID NO: 9 - protein, 8 aa, Homo sapiens
EVYWYFDL

SEQ ID NO: 10 - protein, 11 aa, Homo sapiens
RASQSVSSYLA

SEQ ID NO: 11 - protein, 11 aa, Homo sapiens
RASQSVSSYLA

SEQ ID NO: 12 - protein, 11 aa, Homo sapiens
RASQSVSSYLA

SEQ ID NO: 13 - protein, 7 aa, Homo sapiens
DASNRAT

SEQ ID NO: 14 - protein, 7 aa, Homo sapiens
DASNRAT

SEQ ID NO: 15 - protein, 7 aa, Homo sapiens
DASNRAT

SEQ ID NO: 16 - protein, 10 aa, Homo sapiens
QQLNNWPPYT

SEQ ID NO: 17 - protein, 10 aa, Homo sapiens
QQRNNWPPWT

SEQ ID NO: 18 - protein, 9 aa, Homo sapiens
QQRSNWPPT

SEQ ID NO: 19 - protein, 117 aa, Homo sapiens
QVQLVESGGGVVQPGRSLRLSCAASGFTFSDYGMHWVRQAPGKGLEWVAVIWYDGNNKYYADSVKGRFTISRDNSKNTLYLQMNSLRAEDTAVYYCARDWGRAFDIWGQGTMVTVSS

SEQ ID NO: 20 - protein, 116 aa, Homo sapiens
QVQVVESGGGVVQPGRSLRLSCAASGFTFSNYGMHWVRQAPGKGLEWVAGIWYDGSNKYYADSVKGRFTISRDNSKNTLYLQMNSLRAEDTAVYYCARESPNFDYWGQGTLVTVSS

SEQ ID NO: 21 - protein, 116 aa, Homo sapiens
DVHLVQSGGGLVHPGGSLRLSCAGSGFTFSTYTMHWIRQAPGKDLEWVSAIGTGGGTDYADSVKGRFTISRDNAKNSLYLQMNSLRAEDMAVYYCAREVYWYFDLWGRGTLVTVSS

SEQ ID NO: 22 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFALYYCQQLNNWPPYTFGQGTKLEIK

SEQ ID NO: 23 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRNNWPPWTFGQGTKVEIK

SEQ ID NO: 24 - protein, 107 aa, Homo sapiens [sequence omitted]

SEQ ID NO: 25 - DNA, 348 bp, Homo sapiens
caggtgcagctggtggagtctgggggaggcgtggtccagcctgggaggtccctgagactctcctgtgcagcgtctggattcaccttcagtgactatggcatgcactgggtccgccaggctccaggcaaggggctggagtgggtggcagttatatggtatggaaataataaatactatgcagactccgtgaagggccgattcaccatctccagagacaattccaagaacacgctgtatctgcaaatgaacagtctgagagccgaggacacggctgtgtattactgtgcgagggactggggacgggcttttgatatctggggccaagggacaatggtcaccgtctcttca

SEQ ID NO: 26 - DNA, 348 bp, Homo sapiens
caggtgcaggtggtggagtctgggggaggcgtggtccagcctgggaggtccctgagactctcctgtgcagcgtctggattcaccttcagtaactatggcatgcactgggtccgccaggctccaggcaaggggctggagtgggtggcaggtatatggtatgatggaagtaataaatactatgcagactccgtgaagggccgattcaccatctccagagacaattccaagaacacgctgtatctgcaaatgaacagcctgagagccgaggacacggctgtgtattactgtgcgagagaaagccccaactttgactactggggccagggaaccctggtcaccgtctcctca

SEQ ID NO: 27 - DNA, 348 bp, Homo sapiens
gatgttcatctggtgcagtctgggggaggcttggtacatcctggggggtccctgagactctcctgtgcaggctctggattcaccttcagtacctatacaatgcactggattcgccaggctccaggaaaagatctggagtgggtatcagctattggtactggtggtggcacagactatgcagactccgtgaagggccgattcaccatctccagagacaatgccaagaactccttgtatcttcaaatgaacagcctgagagccgaggacatggctgtgtattactgtgcaagagaggtctactggtacttcgatctctggggccgtggcaccctggtcactgtctcctca

SEQ ID NO: 28 - DNA, 324 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcactttattactgtcagcagcttaacaactggcctccgtacacttttggccaggggaccaagctggagatcaaa

SEQ ID NO: 29 - DNA, 324 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaacaactggcctccgtggacgttcggccaagggaccaaggtggaaatcaaa

SEQ ID NO: 30 - DNA, 321 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaa

SEQ ID NO: 31 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 32 - protein, 97 aa, Homo sapiens
EVQLVQSGGGLVHPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 33 - protein, 94 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNW

SEQ ID NO: 34 - protein, 97 aa, Homo sapiens
VQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 35 - protein, 97 aa, Homo sapiens
VQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 36 - protein, 116 aa, Homo sapiens
  MISC_FEATURE (13)..(13): Xaa is Lys or Gln
DVHLVQSGGGLVXPGGSLRLSCAGSGFTFSTYTMHWIRQAPGKDLEWVSAIGTGGGTDYADSVKGRFTISRDNAKNSLYLQMNSLRAEDTAVYYCAREVYWYFDLWGRGTLVTVSS

SEQ ID NO: 37 - protein, 429 aa, Homo sapiens
MLPRLLLLICAPLCEPAELFLIASPSHPTEGSPVTLTCKMPFLQSSDAQFQFCFFRDTRALGPGWSSSPKLQIAAMWKEDTGSYWCEAQTMASKVLRSRRSQINVHRVPVADVSLETQPPGGQVMEGDRLVLICSVAMGTGDITFLWYKGAVGLNLQSKTQRSLTAEYEIPSVRESDAEQYYCVAENGYGPSPSGLVSITVRIPVSRPILMLRAPRAQAAVEDVLELHCEALRGSPPILYWFYHEDITLGSRSAPSGGGASFNLSLTEEHSGNYSCEANNGLGAQRSEAVTLNFTVPTGARSNHLTSGVIEGLLSTLGPATVALLFCYGLKRKIGRRSARDPLRSLPSPLPQEFTYLNSPTPGQLQPIYENVNVVSGDEVYSLAYYNQPEQESVAAETLGTHMEDKVSLDIYSRLRKANITDVDYEDAM

SEQ ID NO: 38 - protein, 515 aa, Homo sapiens
MLLWASLLAFAPVCGQSAAAHKPVISVHPPWTTFFKGERVTLTCNGFQFYATEKTTWYHRHYWGEKLTLTPGNTLEVRESGLYRCQARGSPRSNPVRLLFSSDSLILQAPYSVFEGDTLVLRCHRRRKEKLTAVKYTWNGNILSISNKSWDLLIPQASSNNNGNYRCIGYGDENDVFRSNFKIIKIQELFPHPELKATDSQPTEGNSVNLSCETQLPPERSDTPLHFNFFRDGEVILSDWSTYPELQLPTVWRENSGSYWCGAETVRGNIHKHSPSLQIHVQRIPVSGVLLETQPSGGQAVEGEMLVLVCSVAEGTGDTTFSWHREDMQESLGRKTQRSLRAELELPAIRQSHAGGYYCTADNSYGPVQSMVLNVTVRETPGNRDGLVAAGATGGLLSALLLAVALLFHCWRRRKSGVGFLGDETRLPPAPGPGESSHSICPAQVELQSLYVDVHPKKGDLVYSEIQTTQLGEEEEANTSRTLLEDKDVSVVYSEVKTQHPDNSAGKISSKDEES

SEQ ID NO: 39 - protein, 977 aa, Homo sapiens
MLLWVILLVLAPVSGQFARTPRPIIFLQPPWTTVFQGERVTLTCKGFRFYSPQKTKWYHRYLGKEILRETPDNILEVQESGEYRCQAQGSPLSSPVHLDFSSASLILQAPLSVFEGDSVVLRCRAKAEVTLNNTIYKNDNVLAFLNKRTDFHIPHACLKDNGAYRCTGYKESCCPVSSNTVKIQVQEPFTRPVLRASSFQPISGNPVTLTCETQLSLERSDVPLRFRFFRDDQTLGLGWSLSPNFQITAMWSKDSGFYWCKAATMPHSVISDSPRSWIQVQIPASHPVLTLSPEKALNFEGTKVTLHCETQEDSLRTLYRFYHEGVPLRHKSVRCERGASISFSLTTENSGNYYCTADNGLGAKPSKAVSLSVTVPVSHPVLNLSSPEDLIFEGAKVTLHCEAQRGSLPILYQFHHEDAALERRSANSAGGVAISFSLTAEHSGNYYCTADNGFGPQRSKAVSLSITVPVSHPVLTLSSAEALTFEGATVTLHCEVQRGSPQILYQFYHEDMPLWSSSTPSVGRVSFSFSLTEGHSGNYYCTADNGFGPQRSEVVSLFVTVPVSRPILTLRVPRAQAVVGDLLELHCEAPRGSPPILYWFYHEDVTLGSSSAPSGGEASFNLSLTAEHSGNYSCEANNGLVAQHSDTISLSVIVPVSRPILTFRAPRAQAVVGDLLELHCEALRGSSPILYWFYHEDVTLGKISAPSGGGASFNLSLTTEHSGIYSCEADNGPEAQRSEMVTLKVAVPVSRPVLTLRAPGTHAAVGDLLELHCEALRGSPLILYRFFHEDVTLGNRSSPSGGASLNLSLTAEHSGNYSCEADNGLGAQRSETVTLYITGLTANRSGPFATGVAGGLLSIAGLAAGALLLYCWLSRKAGRKPASDPARSPPDSDSQEPTYHNVPAWEELQPVYTNANPRGENVVYSEVRIIQEKKKHAVASDPRHLRNKGSPIIYSEVKVASTPVSGSLFLASSAPHR

SEQ ID NO: 40 - protein, 734 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 41 - protein, 508 aa, Homo sapiens
MLLWSLLVIFDAVTEQADSLTLVAPSSVFEGDSIVLKCQGEQNWKIQKMAYHKDNKELSVFKKFSDFLIQSAVLSDSGNYFCSTKGQLFLWDKTSNIVKIKVQELFQRPVLTASSFQPIEGGPVSLKCETRLSPQRLDVQLQFCFFRENQVLGSGWSSSPELQISAVWSEDTGSYWCKAETVTHRIRKQSLQSQIHVQRIPISNVSLEIRAPGGQVTEGQKLILLCSVAGGTGNVTFSWYREATGTSMGKKTQRSLSAELEIPAVKESDAGKYYCRADNGHVPIQSKVVNIPVRIPVSRPVLTLRSPGAQAAVGDLLELHCEALRGSPPILYQFYHEDVTLGNSSAPSGGGASFNLSLTAEHSGNYSCEANNGLGAQCSEAVPVSISGPDGYRRDLMTAGVLWGLFGVLGFTGVALLLYALFHKISGESSATNEPRGASRPNPQEFTYSSPTPDMEELQPVYVNVGSVDVDVVYSQVWSMQQPESSANIRTLLENKDSQVIYSSVKKS

(19) United States
(12) Patent Application Publication
(10) Pub. No.: US 2008/0187547 A1
(43) Pub. Date: Aug. 7, 2008

(54) IRTA-5 ANTIBODIES AND THEIR USES

(76) Inventors: Robert Graziano, Frenchtown, NJ (US); Josephine M. Cardarelli, San Carlos, CA (US); Thomas D. Kempe, Sunnyvale, CA (US); Beth Cutter, Livermore, CA (US); Mohan Srinivasan, San Jose, CA (US)

Correspondence Address: BAKER BOTTS L.L.P., 30 ROCKEFELLER PLAZA, 44th Floor, NEW YORK, NY, 10112-4498, US

(21) Appl. No.: 11/958,683

(22) Filed: Dec. 18, 2007

Related U.S. Application Data
(63) Continuation of application No. 11/093,274, filed on Mar. 28, 2005, now abandoned.
(60) Provisional application No. 60/557,741, filed on Mar. 29, 2004.

(51) Int. Cl. A61K 39/395; C07K 16/18; A61P 43/00

(52) U.S. Cl. 424/172.1; 530/387.9; 530/387.3; 530/387.7

(57) ABSTRACT

The present invention provides isolated monoclonal antibodies, particularly human monoclonal antibodies, that specifically bind to IRTA-5 with high affinity. Nucleic acid molecules encoding the antibodies of the invention, expression vectors, host cells and methods for expressing the antibodies of the invention are also provided. Immunoconjugates, bispecific molecules and pharmaceutical compositions comprising the antibodies of the invention are also provided. The invention also provides methods for detecting IRTA-5, as well as methods for treating various B cell malignancies, including non-Hodgkin's lymphoma.

### CROSS-REFERENCE TO RELATED APPLICATIONS

This application is a continuation of U.S. patent application Ser. No. 11/093,274, filed Mar. 28, 2005, which claims priority to U.S. Provisional Patent Application Ser. No. 60/557,741, filed Mar. 29, 2004, the contents of which are hereby incorporated in their entirety.

### BACKGROUND OF THE INVENTION

The Immune Receptor Translocation Associated (IRTA) genes/proteins, also known as Fc Receptor Homolog (FcRH) genes, consist of a five-member family of immunoglobulin-like cell surface receptors (Miller et al., (2002) Blood. 99:2662; Davis et al., (2002) Immunological Reviews. 190:123). The IRTAs were initially discovered by analysis of the breakpoints of a multiple myeloma cell line which contained a 1q21 chromosomal rearrangement (Hatzivassiliou et al., (2001) Immunity. 14:277). Each of the IRTA glycoproteins contains between 3 to 9 extracellular Ig-like domains (Miller, 2002, supra). IRTAs are also characterized by having a cytoplasmic domain containing 3 to 5 tyrosine residues contained within particular motifs, suggesting the presence of immunotyrosine inhibitory motifs (ITIM) and immunotyrosine activation-like (ITAM-like) motifs (Miller, 2002, supra; Hatzivassiliou, 2001, supra).

IRTAs are expressed in peripheral lymphoid tissues, including lymph nodes, tonsils, resting peripheral B cells and normal germinal center B cells (Davis et al., (2001) PNAS. 98:9772). IRTA 2, 3, 4, and 5 are all expressed at high levels in spleen, whereas, by comparison, IRTA1 has been detected in lower levels in the spleen. IRTA expression has been analyzed within the B cell compartment of human tonsil tissue. IRTA 1 is expressed outside of lymphoid follicles in the marginal zone pattern and in intraepithelial lymphocytes. IRTA2 and 3 are expressed within the germinal center, with highest expression in the centocyte-rich light zone. IRTA4 and 5 are expressed highest within mantle zones, indicating expression in naïve B cells. (Miller, 2002, supra) IRTA5 is unique among the IRTAs in that it has a charged glutamic acid residue in the transmembrane region, suggesting it may heterodimerize with a protein containing a positively charged amino acid in a nearby position, as is the case for many ITAM-bearing proteins (Miller, 2002, supra).

The IRTA genes have been shown to be highly expressed in B cell non-Hodgkin's lymphoma, chronic lymphocytic leukemias, follicular lymphomas, diffuse large cell lymphomas of B lineage, and multiple myelomas (Davis, 2001, supra).

### SUMMARY OF THE INVENTION

The present invention provides isolated monoclonal antibodies, in particular human monoclonal antibodies, that bind to IRTA-5 and that exhibit numerous desirable properties. These properties include high affinity binding to human IRTA-5, but lacking substantial cross-reactivity with either human IRTA-1, IRTA-2, IRTA-3, or IRTA-4. Furthermore, the antibodies bind specifically to B cells. Still further, antibodies of the invention have been shown to bind to B cell tumor cell lines but not to T cells, dendritic cells, monocytes or natural killer cells.

In preferred embodiments of the invention, the human IRTA-5 comprises a polypeptide having an amino acid sequence as set forth in SEQ ID NO: 37 [Genbank Acc. No. AAL60250]; the human IRTA-1 comprises a polypeptide having an amino acid sequence as set forth in SEQ ID NO: 38 [Genbank Acc. No. NP_{—}112572]; the human IRTA-2 comprises a polypeptide having an amino acid sequence as set forth in SEQ ID NO: 39 [Genbank Acc. No. NP_{—}112571]; the human IRTA-3 comprises a polypeptide having an amino acid sequence as set forth in SEQ ID NO: 40 [Genbank Acc. No. AAL59390]; and/or the human IRTA-4 comprises a polypeptide having an amino acid sequence as set forth in SEQ ID NO: 41 [Genbank Acc. No. AAL60249].

In one aspect, the invention pertains to an isolated monoclonal antibody, or an antigen-binding portion thereof, wherein the antibody:
(a) binds to human IRTA-5 with a K_{D} of 5×10⁻⁸ M or less;
(b) does not substantially bind to human IRTA-1, IRTA-2, IRTA-3 and IRTA-4; and
(c) binds to human B lymphocytes and to B cell tumor lines but does not substantially bind to CD3+ peripheral blood T cells, CD1A+ peripheral blood dendritic cells, CD14+ peripheral blood monocytes, or CD56+ peripheral blood natural killer cells.
   Preferably the antibody is a human antibody, although in alternative embodiments the antibody can be a murine antibody, a chimeric antibody or humanized antibody.

In more preferred embodiments, the antibody binds to human IRTA-5 with a K_{D} of 3×10⁻⁸ M or less, binds to human IRTA-5 with a K_{D} of 1×10⁻⁹ M or less, binds to human IRTA-5 with a K_{D} of 0.1×10⁻⁹ M or less, binds to human IRTA-5 with a K_{D} of 0.05×10⁻⁹ M or less or binds to human IRTA-5 with a K_{D} of between 1×10⁻⁹ and 1×10⁻¹¹ M.

In another preferred embodiment, the B cell tumor lines are selected from the group consisting of Daudi, Ramos, and SU-DHL-4 cell lines.

In another embodiment, the invention provides an isolated monoclonal antibody, or antigen binding portion thereof, wherein the antibody cross-competes for binding to IRTA-5 with a reference antibody comprising:
(a) a heavy chain variable region comprising an amino acid sequence selected from the group consisting of SEQ ID NOs: 19, 20, and 21; and
(b) a light chain variable region comprising an amino acid sequence selected from the group consisting of SEQ ID NOs: 22, 23, and 24.
   In various embodiments, the reference antibody comprises:
(a) a heavy chain variable region comprising the amino acid sequence of SEQ ID NO: 19; and
(b) a light chain variable region comprising the amino acid sequence of SEQ ID NO: 22;
   or the reference antibody comprises:
(a) a heavy chain variable region comprising the amino acid sequence of SEQ ID NO: 20; and
(b) a light chain variable region comprising the amino acid sequence of SEQ ID NO: 23;
   or the reference antibody comprises:
(a) a heavy chain variable region comprising the amino acid sequence of SEQ ID NO: 21; and
(b) a light chain variable region comprising the amino acid sequence of SEQ ID NO: 24.

In another aspect, the invention pertains to an isolated monoclonal antibody, or an antigen-binding portion thereof, comprising a heavy chain variable region that is the product of or derived from a human V_{H} 3-33 gene, wherein the antibody specifically binds IRTA-5. The invention also provides an isolated monoclonal antibody, or an antigen-binding portion thereof, comprising a heavy chain variable region that is the product of or derived from a human V_{H} DP44 gene, a human V_{H} 3-23 gene or a human V_{H} 3-7 gene, wherein the antibody specifically binds IRTA-5. The invention still further provides an isolated monoclonal antibody, or an antigen-binding portion thereof, comprising a light chain variable region that is the product of or derived from a human V_{K} L6 gene, wherein the antibody specifically binds IRTA-5.

In a preferred embodiment, the invention provides an isolated monoclonal antibody, or an antigen-binding portion thereof, comprising:
(a) a heavy chain variable region of a human V_{H} 3-33, V_{H} DP44, V_{H} 3-23, or V_{H} 3-7 gene; and
(b) a light chain variable region of a human Vk L6;
wherein the antibody specifically binds to IRTA-5.
   In a preferred embodiment, the antibody comprises a heavy chain variable region of a human V_{H} 3-33 gene and a light chain variable region of a human V_{K} L6 gene. In another preferred embodiment, the antibody comprises a heavy chain variable region of a human V_{H} DP44 gene and a light chain variable region of a human V_{K} L6 gene.

In another aspect, the invention provides an isolated monoclonal antibody, or antigen binding portion thereof, comprising:
a heavy chain variable region that comprises CDR1, CDR2, and CDR3 sequences; and a light chain variable region that comprises CDR1, CDR2, and CDR3 sequences, wherein:
(a) the heavy chain variable region CDR3 sequence comprises an amino acid sequence selected from the group consisting of amino acid sequences of SEQ ID NOs: 7, 8, and 9, and conservative modifications thereof;
(b) the light chain variable region CDR3 sequence comprises an amino acid sequence selected from the group consisting of amino acid sequence of SEQ ID NOs: 16, 17, and 18, and conservative modifications thereof;
(c) the antibody binds to human IRTA-5 with a K_{D} of 5×10⁻⁸ M or less;
(d) the antibody does not substantially bind to human IRTA-1, IRTA-2, IRTA-3 and IRTA-4; and
(e) the antibody binds to human B lymphocytes and to B cell tumor lines but does not substantially bind to CD3+ peripheral blood T cells, CD1A+ peripheral blood dendritic cells, CD14+ peripheral blood monocytes, or CD56+ peripheral blood natural killer cells.
   Preferably, the heavy chain variable region CDR2 sequence comprises an amino acid sequence selected from the group consisting of amino acid sequences of SEQ ID NOs: 4, 5, and 6, and conservative modifications thereof; and the light chain variable region CDR2 sequence comprises an amino acid sequence selected from the group consisting of amino acid sequences of SEQ ID NOs: 13, 14, and 15, and conservative modifications thereof. Preferably, the heavy chain variable region CDR1 sequence comprises an amino acid sequence selected from the group consisting of amino acid sequences of SEQ ID NOs: 1, 2, and 3, and conservative modifications thereof; and the light chain variable region CDR1 sequence comprises an amino acid sequence selected from the group consisting of amino acid sequences of SEQ ID NOs: 10, 11, and 12, and conservative modifications thereof.

In a preferred embodiment, the B cell tumor lines are selected from the group consisting of Daudi, Ramos, and SU-DHL-4 cell lines.

In yet another aspect, the invention provides an isolated monoclonal antibody, or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NOs: 19, 20, and 21;
(b) the light chain variable region comprises an amino acid sequence that is at least 80% homologous to an amino acid sequence selected from the group consisting of SEQ ID NOs:22, 23, and 24;
(c) the antibody binds to human IRTA-5 with a K_{D} of 5×10⁻⁸ M or less;
(d) the antibody does not substantially bind to human IRTA-1, IRTA-2, IRTA-3 and IRTA-4; and
(e) the antibody binds to human B lymphocytes and to B cell tumor lines but does not substantially bind to CD3+ peripheral blood T cells, CD1A+ peripheral blood dendritic cells, CD14+ peripheral blood monocytes, or CD56+ peripheral blood natural killer cells.

In preferred embodiments, the invention provides an isolated monoclonal antibody, or antigen binding portion thereof, comprising:
(a) a heavy chain variable region CDR1 comprising an amino acid sequence selected from the group consisting of SEQ ID NOs: 1, 2, and 3;
(b) a heavy chain variable region CDR2 comprising an amino acid sequence selected from the group consisting of SEQ ID NOs: 4, 5, and 6;
(c) a heavy chain variable region CDR3 comprising an amino acid sequence selected from the group consisting of SEQ ID NOs: 7, 8, and 9;
(d) a light chain variable region CDR1 comprising an amino acid sequence selected from the group consisting of SEQ ID NOs: 10, 11, and 12;
(e) a light chain variable region CDR2 comprising an amino acid sequence selected from the group consisting of SEQ ID NOs: 13, 14, and 15; and
(f) a light chain variable region CDR3 comprising an amino acid sequence selected from the group consisting of SEQ ID NOs: 16, 17, and 18;
wherein the antibody specifically binds IRTA-5.
   A preferred combination comprises:
(a) a heavy chain variable region CDR1 comprising SEQ ID NO: 1;
(b) a heavy chain variable region CDR2 comprising SEQ ID NO: 4;
(c) a heavy chain variable region CDR3 comprising SEQ ID NO: 7;
(d) a light chain variable region CDR1 comprising SEQ ID NO: 10;
(e) a light chain variable region CDR2 comprising SEQ ID NO: 13; and
(f) a light chain variable region CDR3 comprising SEQ ID NO: 16.
   Another preferred combination comprises:
(a) a heavy chain variable region CDR1 comprising SEQ ID NO: 2;
(b) a heavy chain variable region CDR2 comprising SEQ ID NO: 5;
(c) a heavy chain variable region CDR3 comprising SEQ ID NO: 8;
(d) a light chain variable region CDR1 comprising SEQ ID NO: 11;
(e) a light chain variable region CDR2 comprising SEQ ID NO: 14; and
(f) a light chain variable region CDR3 comprising SEQ ID NO: 17.
   Yet another preferred combination comprises:
(a) a heavy chain variable region CDR1 comprising SEQ ID NO: 3;
(b) a heavy chain variable region CDR2 comprising SEQ ID NO: 6;
(c) a heavy chain variable region CDR3 comprising SEQ ID NO: 9;
(d) a light chain variable region CDR1 comprising SEQ ID NO: 12;
(e) a light chain variable region CDR2 comprising SEQ ID NO: 15; and
(f) a light chain variable region CDR3 comprising SEQ ID NO: 18.

In another preferred embodiment, the B cell tumor lines are selected from the group consisting of Daudi, Ramos, and SU-DHL-4 cell lines.

Other preferred antibodies of the invention, or antigen binding portions thereof comprise:
(a) a heavy chain variable region comprising an amino acid sequence selected from the group consisting of SEQ ID NOs: 19, 20, 21 and 36; and
(b) a light chain variable region comprising an amino acid sequence selected from the group consisting of SEQ ID NOs: 22, 23, and 24;
wherein the antibody specifically binds IRTA-5.
   A preferred combination comprises:
(a) a heavy chain variable region comprising the amino acid sequence of SEQ ID NO: 19; and
(b) a light chain variable region comprising the amino acid sequence of SEQ ID NO: 22.
   Another preferred combination comprises:
(a) a heavy chain variable region comprising the amino acid sequence of SEQ ID NO: 20; and
(b) a light chain variable region comprising the amino acid sequence of SEQ ID NO: 23.
   Yet another preferred combinations comprises:
(a) a heavy chain variable region comprising the amino acid sequence of SEQ ID NO: 21 or 36; and
(b) a light chain variable region comprising the amino acid sequence of SEQ ID NO: 24.

In another aspect of the invention, antibodies, or antigen-binding portions thereof, are provided that compete for binding to IRTA-5 with any of the aforementioned antibodies.

The antibodies of the invention can be, for example, full-length antibodies, for example of an IgG1 or IgG4 isotype. Alternatively, the antibodies can be antibody fragments, such as Fab or Fab′2 fragments, or single chain antibodies.

The invention also provides an immunoconjugate comprising an antibody of the invention, or antigen-binding portion thereof, linked to a therapeutic agent, such as a cytotoxin or a radioactive isotope. The invention also provides a bispecific molecule comprising an antibody, or antigen-binding portion thereof, of the invention, linked to a second functional moiety having a different binding specificity than said antibody, or antigen binding portion thereof.

Compositions comprising an antibody, or antigen-binding portion thereof, or immunoconjugate or bispecific molecule of the invention and a pharmaceutically acceptable carrier are also provided.

Nucleic acid molecules encoding the antibodies, or antigen-binding portions thereof, of the invention are also encompassed by the invention, as well as expression vectors comprising such nucleic acids and host cells comprising such expression vectors. Moreover, the invention provides a transgenic mouse comprising human immunoglobulin heavy and light chain transgenes, wherein the mouse expresses an antibody of the invention, as well as hybridomas prepared from such a mouse, wherein the hybridoma produces the antibody of the invention.

In yet another aspect, the invention provides a method of treating a B cell malignancy in a subject in need of treatment comprising administering to the subject the antibody, or antigen-binding portion thereof, of the invention, such that the B cell malignancy in the subject is treated. The disease can be, for example, non-Hodgkin's lymphoma, chronic lymphocytic leukemias, follicular lymphomas, diffuse large cell lymphomas of B lineage, and multiple myelomas.

The invention also provides methods for making “second generation” anti-IRTA-5 antibodies based on the sequences of the anti-IRTA-5 antibodies provided herein. For example, the invention provides a method for preparing an anti-IRTA-5 antibody comprising:

(a) providing: (i) a heavy chain variable region antibody sequence comprising a CDR1 sequence selected from the group consisting of SEQ ID NOs: 1, 2, and 3, a CDR2 sequence selected from the group consisting of SEQ ID NOs: 4, 5, and 6 and/or a CDR3 sequence selected from the group consisting of SEQ ID NOs: 7, 8, and 9; and/or (ii) a light chain variable region antibody sequence comprising a CDR1 sequence selected from the group consisting of SEQ ID NOs: 10, 11, and 12, a CDR2 sequence selected from the group consisting of SEQ ID NOs: 13, 14, and 15 and/or a CDR3 sequence selected from the group consisting of SEQ ID NOs: 16, 17, and 18;

(b) altering at least one amino acid residue within the heavy chain variable region antibody sequence and/or the light chain variable region antibody sequence to create at least one altered antibody sequence; and

(c) expressing the altered antibody sequence as a protein.

Other features and advantages of the instant invention will be apparent from the following detailed description and examples which should not be construed as limiting. The contents of all references, Genbank entries, patents and published patent applications cited throughout this application are expressly incorporated herein by reference.

### BRIEF DESCRIPTION OF THE DRAWINGS

FIG. 1A shows the nucleotide sequence (SEQ ID NO: 25) and amino acid sequence (SEQ ID NO: 19) of the heavy chain variable region of the 2G5 human monoclonal antibody. The CDR1 (SEQ ID NO: 1), CDR2 (SEQ ID NO: 4) and CDR3 (SEQ ID NO: 7) regions are delineated and the V, D and J germline derivations are indicated.

FIG. 1B shows the nucleotide sequence (SEQ ID NO: 28) and amino acid sequence (SEQ ID NO: 22) of the light chain variable region of the 2G5 human monoclonal antibody. The CDR1 (SEQ ID NO: 10), CDR2 (SEQ ID NO: 13) and CDR3 (SEQ ID NO: 16) regions are delineated and the V and J germline derivations are indicated.

FIG. 2A shows the nucleotide sequence (SEQ ID NO: 26) and amino acid sequence (SEQ ID NO: 20) of the heavy chain variable region of the 5A2 human monoclonal antibody. The CDR1 (SEQ ID NO: 2), CDR2 (SEQ ID NO: 5) and CDR3 (SEQ ID NO: 8) regions are delineated and the V and J germline derivations are indicated.

FIG. 2B shows the nucleotide sequence (SEQ ID NO: 29) and amino acid sequence (SEQ ID NO: 23) of the light chain variable region of the 5A2 human monoclonal antibody. The CDR1 (SEQ ID NO: 11), CDR2 (SEQ ID NO: 14) and CDR3 (SEQ ID NO: 17) regions are delineated and the V and J germline derivations are indicated.

FIG. 3A shows the nucleotide sequence (SEQ ID NO: 27) and amino acid sequence (SEQ ID NO: 21) of the heavy chain variable region of the 7G8 human monoclonal antibody. The CDR1 (SEQ ID NO: 3), CDR2 (SEQ ID NO: 6) and CDR3 (SEQ ID NO: 9) regions are delineated and the V and J germline derivations are indicated.

FIG. 3B shows the nucleotide sequence (SEQ ID NO: 30) and amino acid sequence (SEQ ID NO: 24) of the light chain variable region of the 7G8 human monoclonal antibody. The CDR1 (SEQ ID NO: 12), CDR2 (SEQ ID NO: 15) and CDR3 (SEQ ID NO: 18) regions are delineated and the V and J germline derivations are indicated.

FIG. 4 shows the alignment of the amino acid sequence of the heavy chain variable region of 2G5 and 5A2 with the human germline V_{H} 3-33 amino acid sequence (SEQ ID NO: 31).

FIG. 5 shows the alignment of the amino acid sequence of the heavy chain variable region of 7G8 with the human germline V_{H} DP44 amino acid sequences (SEQ ID NO: 32).

FIG. 6 shows the alignment of the amino acid sequence of the light chain variable region of 2G5, 5A2, and 7G8 with the human germline Vₖ L6 amino acid sequence (SEQ ID NO:33).

FIG. 7 shows the alignment of the amino acid sequence of the heavy chain variable region of 7G8 (SEQ ID NO: 21) and a mutated form of the heavy chain variable region of 7G8 referred to as 7G8(mut) (SEQ ID NO: 36) with the human germline V_{H} DP44, V_{H} 3-23 and V_{H} 3-7 amino acid sequences (SEQ ID NOs: 32, 34 and 35, respectively)

FIG. 8 shows epitope groupings of anti-IRTA-5 antibodies, based on BIAcore analysis.

FIG. 9 is a graph showing the results of experiments demonstrating that the human monoclonal antibodies, 4B7, 2G1, 7F5, 7G8, 5A2, 1E5, and 2G5, directed against human IRTA-5, specifically bind to human IRTA-5.

FIG. 10A shows the results of flow cytometry experiments demonstrating that the human monoclonal antibodies 2G5 and 7G8, directed against human IRTA-5, bind to CD19+B cells.

FIG. 10B shows the results of flow cytometry experiments demonstrating that the human monoclonal antibodies 2G5 and 7G8, directed against human IRTA-5, do not bind to CD3+ peripheral blood T cells, CD1A+ peripheral blood dendritic cells, CD14+ peripheral blood monocytes, or CD56+ peripheral blood NK cells.

FIG. 11 shows histogram plots demonstrating that the human monoclonal antibody 2G2, directed against human IRTA-5, specifically binds the cell surface of tumor cell lines of B cell origin.

FIG. 12 shows the results of flow cytometry experiments demonstrating binding of the human monoclonal antibody 2G5, directed against human IRTA-5, to the B-cell tumor lines Karpas 1106P, SU-DHL-4, Granta 519, and L-540.

### DETAILED DESCRIPTION OF THE INVENTION

The present invention relates to isolated monoclonal antibodies, particularly human monoclonal antibodies, that bind specifically to IRTA-5 and that inhibit functional properties of IRTA-5. In certain embodiments, the antibodies of the invention are derived from particular heavy and light chain germline sequences and/or comprise particular structural features such as CDR regions comprising particular amino acid sequences. The invention provides isolated antibodies, methods of making such antibodies, immunoconjugates and bispecific molecules comprising such antibodies and pharmaceutical compositions containing the antibodies, immunconjugates or bispecific molecules of the invention. The invention also relates to methods of using the antibodies, such as to detect IRTA-5, as well as to treat diseases associated with expression of IRTA-5, such as B cell malignancies that express IRTA-5. Accordingly, the invention also provides methods of using the anti-IRTA-5 antibodies of the invention to treat B cell malignancies, for example, in the treatment of non-Hodgkin's lymphoma, chronic lymphocytic leukemias, follicular lymphomas, diffuse large cell lymphomas of B lineage, and multiple myelomas.

In order that the present invention may be more readily understood, certain terms are first defined. Additional definitions are set forth throughout the detailed description.

The terms “immunoglobulin superfamily receptor translocation associated gene 5” and “IRTA-5” are used interchangeably, and include variants, isoforms and species homologs of human IRTA-5. Accordingly, human antibodies of the invention may, in certain cases, cross-react with IRTA-5 from species other than human. In other cases, the antibodies may be completely specific for human IRTA-5 and may not exhibit species or other types of cross-reactivity. The complete amino acid sequence of human IRTA-5 has Genbank accession number AAL60250 (SEQ ID NO: 37).

The terms “IRTA-1”, “IRTA-2”, “IRTA-3”, and “IRTA-4” include variants, isoforms and species homologs of human “IRTA-1”, “IRTA-2”, “IRTA-3”, and “IRTA-4”, respectively. The complete amino acid sequence of human IRTA-1 has Genbank accession number NP_{—}112572 (SEQ ID NO: 38). The complete amino acid sequence of human IRTA-2 has Genbank accession number NP_{—}112571 (SEQ ID NO: 39). The complete amino acid sequence of human IRTA-3 has Genbank accession number AAL59390 (SEQ ID NO: 40). The complete amino acid sequence of human IRTA-4 has Genbank accession number AAL60249 (SEQ ID NO: 41).

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present invention is the IRTA-5 receptor.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., IRTA-5). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al, (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds IRTA-5 is substantially free of antibodies that specifically bind antigens other than IRTA-5). An isolated antibody that specifically binds IRTA-5 may, however, have cross-reactivity to other antigens, such as IRTA-5 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human IRTA-5” is intended to refer to an antibody that binds to human IRTA-5 with a K_{D} of 5×10⁻⁸ M or less, more preferably 3×10⁻⁸ M or less, and even more preferably 1×10⁻⁹ M or less.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacoret system.

As used herein, the term “high affinity” for an IgG antibody refers to an antibody having a K_{D} of 10⁻⁸ M or less, more preferably 10⁻⁹ M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, more preferably 10⁻⁸ M or less.

As used herein, the term “subject” includes any human or nonhuman animal. The term “nonhuman animal” includes all vertebrates, e.g., mammals and non-mammals, such as nonhuman primates, sheep, dogs, cats, horses, cows chickens, amphibians, reptiles, etc.

Various aspects of the invention are described in further detail in the following subsections.

### Anti-IRTA-5 Antibodies

The antibodies of the invention including those having the particular germline sequences, homologous antibodies, antibodies with conservative modifications, engineered and modified antibodies are characterized by particular functional features or properties of the antibodies. For example, the antibodies bind specifically to human IRTA-5. Preferably, an antibody of the invention binds to IRTA-5 with high affinity, for example with a K_{D} of 10⁻⁸ M or less or 10⁻⁹ M or less or even 10⁻¹⁰ M or less. The anti-IRTA-5 antibodies of the invention preferably exhibit one or more of the following characteristics:
(a) binds to human IRTA-5 with a K_{D} of 5×10⁻⁸ M or less;
(b) does not substantially bind to human IRTA-1, IRTA-2, IRTA-3 and IRTA-4; and/or
(c) binds to human B lymphocytes and to B cell tumor lines but does not substantially bind to CD3+ peripheral blood T cells, CD1A+ peripheral blood dendritic cells, CD14+ peripheral blood monocytes, or CD56+ peripheral blood natural killer cells.
   More preferably, the antibody binds to human IRTA-5 with a K_{D} of 3×10⁻⁸ M or less, or with a K_{D} of 1×10⁻⁹ M or less, or with a K_{D} of 0.1×10⁻⁹ M or less, or with a K_{D} of 0.05×10⁹ M or less or with a K_{D} of between 1×10⁻⁹ and 1×10⁻¹¹ M.

In a specific embodiment, an anti-IRTA-5 antibody has the characteristics of exemplified antibody 2G5, 5A2, 7G8, 1E5, 7F5, 4B7, or 2G1, as described in the examples. In another embodiment, an anti-IRTA-5 antibody competes with one or more of 2G5, 5A2, 7G8, 1E5, 7F5, 4B7, or 2G1 for binding to IRTA-5.

Standard assays to evaluate the binding ability of the antibodies toward IRTA-5 are known in the art, including for example, ELISAs, Western blots and RIAs. Suitable assays are described in detail in the Examples. The binding kinetics (e.g., binding affinity) of the antibodies also can be assessed by standard assays known in the art, such as by Biacore analysis.

An antibody of the invention does not “substantially bind” to IRTA-1, IRTA-2, IRTA-3, or IRTA-4 when it possesses a selectivity for IRTA-5 over one of the other IRTAs of greater than about 10:1, and preferably greater than about 100:1. Selectivity can be measured by immunoassay.

### Monoclonal Antibodies 2G5, 5A2, and 7G8

Preferred antibodies of the invention are the human monoclonal antibodies 2G5, 5A2, and 7G8, isolated and structurally characterized as described in Examples 1 and 2. The V_{H} amino acid sequences of 2G5, 5A2, and 7G8 are shown in SEQ ID NOs: 19, 20, and 21, respectively. The V_{L} amino acid sequences of 2G5, 5A2, and 7G8 are shown in SEQ ID NOs: 22, 23, and 24, respectively.

Given that each of these antibodies can bind to IRTA-5, the V_{H} and V_{L} sequences can be “mixed and matched” to create other anti-IRTA-5 binding molecules of the invention. IRTA-5 binding of such “mixed and matched” antibodies can be tested using the binding assays described above and in the Examples (e.g., ELISAs). Preferably, when V_{H} and V_{L} chains are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence. For example, the V_{H} and V_{L} sequences of 2G5 and 5A2 are particularly amenable for mixing and matching, since these antibodies use V_{H} and V_{L} sequences derived from the same germline sequences (V_{H} 3-33 and Vₖ L6) and thus they exhibit structural similarity.

Accordingly, in one aspect, the invention provides an isolated monoclonal antibody, or antigen binding portion thereof comprising:

(a) a heavy chain variable region comprising an amino acid sequence selected from the group consisting of SEQ ID NOs: 19, 20, and 21; and

(b) a light chain variable region comprising an amino acid sequence selected from the group consisting of SEQ ID NOs: 22, 23, and 24;

wherein the antibody specifically binds IRTA-5, preferably human IRTA-5.

Preferred heavy and light chain combinations include:

(a) a heavy chain variable region comprising the amino acid sequence of SEQ ID NO: 19; and (b) a light chain variable region comprising the amino acid sequence of SEQ ID NO: 22; or

(a) a heavy chain variable region comprising the amino acid sequence of SEQ ID NO: 20; and (b) a light chain variable region comprising the amino acid sequence of SEQ ID NO: 23; or

(a) a heavy chain variable region comprising the amino acid sequence of SEQ ID NO: 21; and (b) a light chain variable region comprising the amino acid sequence of SEQ ID NO: 24.

In another aspect, the invention provides antibodies that comprise the heavy chain and light chain CDR1s, CDR2s and CDR3s of 2G5, 5A2, and 7G8, or combinations thereof. The amino acid sequences of the V_{H} CDR1s of 2G5, 5A2, and 7G8 are shown in SEQ ID NOs: 1, 2, and 3. The amino acid sequences of the V_{H} CDR2s of 2G5, 5A2, and 7G8 are shown in SEQ ID NOs: 4, 5, and 6. The amino acid sequences of the V_{H} CDR3s of 2G5, 5A2, and 7G8 are shown in SEQ ID NOs: 7, 8, and 9. The amino acid sequences of the Vₖ CDR1s of 2G5, 5A2, and 7G8 are shown in SEQ ID NOs: 10, 11, and 12. The amino acid sequences of the Vₖ CDR2s of 2G5, 5A2, and 7G8 are shown in SEQ ID NOs: 13, 14, and 15. The amino acid sequences of the Vₖ CDR3s of 2G5, 5A2, and 7G8 are shown in SEQ ID NOs: 16, 17, and 18. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of these antibodies can bind to IRTA-5 and that antigen-binding specificity is provided primarily by the CDR1, CDR2, and CDR3 regions, the V_{H} CDR1, CDR2, and CDR3 sequences and Vₖ CDR1, CDR2, and CDR3 sequences can be “mixed and matched” (i.e., CDRs from different antibodies can be mixed and matched, although each antibody must contain a V_{H} CDR1, CDR2, and CDR3 and a Vₖ CDR1, CDR2, and CDR3) to create other anti-IRTA-5 binding molecules of the invention. IRTA-5 binding of such “mixed and matched” antibodies can be tested using the binding assays described above and in the Examples (e.g., ELISAs, Biacore analysis). Preferably, when V_{H} CDR sequences are mixed and matched, the CDR1, CDR2 and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence preferably is replaced with a structurally similar CDR sequence(s). For example, the V_{H} CDR1s of 2G5, 5A2, and 7G8 share some structural similarity and therefore are amenable to mixing and matching. It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies 2G5, 5A2, and 7G8.

Accordingly, in another aspect, the invention provides an isolated monoclonal antibody, or antigen binding portion thereof comprising:

(a) a heavy chain variable region CDR1 comprising an amino acid sequence selected from the group consisting of SEQ ID NOs: 1, 2, and 3;

(b) a heavy chain variable region CDR2 comprising an amino acid sequence selected from the group consisting of SEQ ID NOs: 4, 5, and 6;

(c) a heavy chain variable region CDR3 comprising an amino acid sequence selected from the group consisting of SEQ ID NOs: 7, 8, and 9;

(d) a light chain variable region CDR1 comprising an amino acid sequence selected from the group consisting of SEQ ID NOs: 10, 11, and 12;

(e) a light chain variable region CDR2 comprising an amino acid sequence selected from the group consisting of SEQ ID NOs: 13, 14, and 15; and

(f) a light chain variable region CDR3 comprising an amino acid sequence selected from the group consisting of SEQ ID NOs: 16, 17, and 18;

wherein the antibody specifically binds IRTA-5, preferably human IRTA-5.

In a preferred embodiment, the antibody comprises:

(a) a heavy chain variable region CDR1 comprising SEQ ID NO: 1;

(b) a heavy chain variable region CDR2 comprising SEQ ID NO: 4;

(c) a heavy chain variable region CDR3 comprising SEQ ID NO: 7;

(d) a light chain variable region CDR1 comprising SEQ ID NO: 10;

(e) a light chain variable region CDR2 comprising SEQ ID NO: 13; and

(f) a light chain variable region CDR3 comprising SEQ ID NO: 16.

In another preferred embodiment, the antibody comprises:

(a) a heavy chain variable region CDR1 comprising SEQ ID NO: 2;

(b) a heavy chain variable region CDR2 comprising SEQ ID NO: 5;

(c) a heavy chain variable region CDR3 comprising SEQ ID NO: 8;

(d) a light chain variable region CDR1 comprising SEQ ID NO: 11;

(e) a light chain variable region CDR2 comprising SEQ ID NO: 14; and

(f) a light chain variable region CDR3 comprising SEQ ID NO: 17.

In another preferred embodiment, the antibody comprises:

(a) a heavy chain variable region CDR1 comprising SEQ ID NO: 3;

(b) a heavy chain variable region CDR2 comprising SEQ ID NO: 6;

(c) a heavy chain variable region CDR3 comprising SEQ ID NO: 9;

(d) a light chain variable region CDR1 comprising SEQ ID NO: 12;

(e) a light chain variable region CDR2 comprising SEQ ID NO: 15; and

(f) a light chain variable region CDR3 comprising SEQ ID NO: 18.

### Antibodies Having Particular Germline Sequences

In certain embodiments, an antibody of the invention comprises a heavy chain variable region from a particular germline heavy chain immunoglobulin gene and/or a light chain variable region from a particular germline light chain immunoglobulin gene.

For example, in a preferred embodiment, the invention provides an isolated monoclonal antibody, or an antigen-binding portion thereof, comprising a heavy chain variable region that is the product of or derived from a human V_{H} 3-33 gene, wherein the antibody specifically binds IRTA-5. In another preferred embodiment, the invention provides an isolated monoclonal antibody, or an antigen-binding portion thereof, comprising a heavy chain variable region that is the product of or derived from a human V_{H} DP44 gene, a human V_{H} 3-23 gene or a human V_{H} 3-7 gene, wherein the antibody specifically binds IRTA-5. In yet another preferred embodiment, the invention provides an isolated monoclonal antibody, or an antigen-binding portion thereof, comprising a light chain variable region that is the product of or derived from a human V_{K} L6 gene, wherein the antibody specifically binds IRTA-5. In yet another preferred embodiment, the invention provides an isolated monoclonal antibody, or antigen-binding portion thereof, wherein the antibody:

(a) comprises a heavy chain variable region that is the product of or derived from a human V_{H} 3-33, V_{H} DP44, V_{H} 3-23, or V_{H} 3-7 gene (which encodes the amino acid sequences set forth in SEQ ID NO: 31, 32, 34 and 36, respectively);

(b) comprises a light chain variable region that is the product of or derived from a human Vₖ L6 gene (which encode the amino acid sequences set forth in SEQ ID NO:33); and

(c) specifically binds to IRTA-5, preferably human IRTA-5.

Examples of antibodies having V_{H} and V_{K} of V_{H} 3-33 and Vₖ L6, respectively, include 2G5 and 5A2. An example of an antibody having V_{H} and V_{K} of V_{H} DP44 and Vk L6, respectively, is 7G8. As discussed in Example 3, given the structural relatedness of V_{H} DP44 to V_{H} 3-23 and V_{H} 3-7, it is expected that other IRTA-5 antibodies of the invention can be selected that utilize a V_{H} region derived from either of these additional germline sequences.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, an antibody of the invention comprises heavy and light chain variable regions comprising amino acid sequences that are homologous to the amino acid sequences of the preferred antibodies described herein, and wherein the antibodies retain the desired functional properties of the anti-IRTA-5 antibodies of the invention.

For example, the invention provides an isolated monoclonal antibody, or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NOs: 19, 20, and 21;
(b) the light chain variable region comprises an amino acid sequence that is at least 80% homologous to an amino acid sequence selected from the group consisting of SEQ ID NOs: 22, 23, and 24;
(c) the antibody binds to human IRTA-5 with a K_{D} of 5×10⁻⁸ M or less;
(d) the antibody does not substantially bind to human IRTA-1, IRTA-2, IRTA-3 and IRTA-4; and
(e) the antibody binds to human B lymphocytes and to B cell tumor lines but does not substantially bind to CD3+ peripheral blood T cells, CD1A+ peripheral blood dendritic cells, CD14+ peripheral blood monocytes, or CD56+ peripheral blood natural killer cells.

In various embodiments, the antibody can be, for example, a human antibody, a humanized antibody or a chimeric antibody.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs: 25, 26, 27, 28, 29, or 30, followed by testing of the encoded altered antibody for retained function (i.e., the functions set forth in (c) and (d) above) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 2G5, 5A2, or 7G8), or conservative modifications thereof, and wherein the antibodies retain the desired functional properties of the anti-IRTA-5 antibodies of the invention. Accordingly, the invention provides an isolated monoclonal antibody, or antigen binding portion thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from the group consisting of amino acid sequences of SEQ ID NOs: 7, 8, and 9, and conservative modifications thereof;
(b) the light chain variable region CDR3 sequence comprises an amino acid sequence selected from the group consisting of amino acid sequence of SEQ ID NOs: 16, 17, and 18, and conservative modifications thereof;
(c) the antibody binds to human IRTA-5 with a K_{D} of 5×10⁻⁸ M or less;
(d) the antibody does not substantially bind to human IRTA-1, IRTA-2, IRTA-3 and IRTA-4; and
(e) the antibody binds to human B lymphocytes and to B cell tumor lines but does not substantially bind to CD3+ peripheral blood T cells, CD1A+ peripheral blood dendritic cells, CD14+ peripheral blood monocytes, or CD56+ peripheral blood natural killer cells.

In a preferred embodiment, the heavy chain variable region CDR2 sequence comprises an amino acid sequence selected from the group consisting of amino acid sequences of SEQ ID NOs: 4, 5, and 6, and conservative modifications thereof; and the light chain variable region CDR2 sequence comprises an amino acid sequence selected from the group consisting of amino acid sequences of SEQ ID NOs: 13, 14, and 15, and conservative modifications thereof. In another preferred embodiment, the heavy chain variable region CDR1 sequence comprises an amino acid sequence selected from the group consisting of amino acid sequences of SEQ ID NOs: 1, 2, and 3, and conservative modifications thereof; and the light chain variable region CDR1 sequence comprises an amino acid sequence selected from the group consisting of amino acid sequences of SEQ ID NOs: 10, 11, and 12, and conservative modifications thereof.

In various embodiments, the antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (c) through (j) above) using the functional assays described herein.

Antibodies that Bind to the Same Epitope as Anti-IRTA-5 Antibodies of the Invention

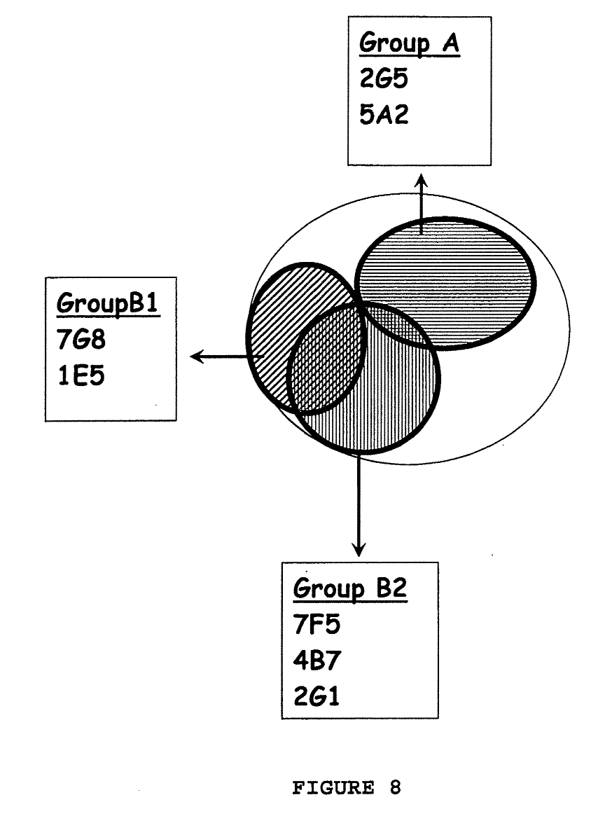

In another embodiment, the invention provides antibodies that bind to the same epitope on human IRTA-5 as any of the IRTA-5 monoclonal antibodies of the invention (i.e., antibodies that have the ability to cross-compete for binding to IRTA-5 with any of the monoclonal antibodies of the invention). Epitope mapping of seven anti-IRTA-5 antibodies (2G5, 5A2, 7G8, 4B7, 7F5, 4B7 and 2G1) has been determined by Biacore analysis (see Example 4) and the antibodies have been shown to fall into three epitope groups, illustrated schematically in FIG. 8. The invention covers anti-IRTA5 antibodies that fall within any of these epitope groups, which can be determined by cross-competition studies with the above-identified antibodies. In preferred embodiments, the reference antibody for cross-competition studies can be the monoclonal antibody 2G5 (having V_{H} and V_{L} sequences as shown in SEQ ID NOs: 19 and 22), the monoclonal antibody 5A2 (having V_{H} and V_{L} sequences as shown in SEQ ID NOs: 20 and 23), or the monoclonal antibody 7G8 (having V_{H} and V_{L} sequences as shown in SEQ ID NOs: 21 and 24). Such cross-competing antibodies can be identified based on their ability to cross-compete with 2G5, 5A2, or 7G8 in standard IRTA-5 binding assays. For example, BIAcore analysis, ELISA assays or flow cytometry may be used to demonstrate cross-competition with the antibodies of the current invention. The ability of a test antibody to inhibit the binding of, for example, 2G5, 5A2, or 7G8, to human IRTA-5 demonstrates that the test antibody can compete with 2G5, 5A2, or 7G8 for binding to human IRTA-5 and thus binds to the same epitope on human IRTA-5 as 2G5, 5A2, or 7G8. In a preferred embodiment, the antibody that binds to the same epitope on human IRTA-5 as 2G5, 5A2, or 7G8 is a human monoclonal antibody. Such human monoclonal antibodies can be prepared and isolated as described in the Examples.

### Engineered and Modified Antibodies

An antibody of the invention further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the invention pertains to an isolated monoclonal antibody, or antigen binding portion thereof, comprising a heavy chain variable region comprising CDR1, CDR2, and CDR3 sequences comprising an amino acid sequence selected from the group consisting of SEQ ID NOs: 1, 2, and 3, SEQ ID NOs: 4, 5, and 6 and SEQ ID NOs: 7, 8, and 9, respectively, and a light chain variable region comprising CDR1, CDR2, and CDR3 sequences comprising an amino acid sequence selected from the group consisting of SEQ ID NOs: 10, 11, and 12, SEQ ID NOs: 13, 14, and 15 and SEQ ID NOs: 16, 17, and 18, respectively. Thus, such antibodies contain the V_{H} and V_{L} CDR sequences of monoclonal antibodies 2G5, 5A2, or 7G8 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference.

Preferred framework sequences for use in the antibodies of the invention are those that are structurally similar to the framework sequences used by selected antibodies of the invention, e.g., similar to the V_{H} 3-33 sequences (SEQ ID NO: 31) and/or the V_{H} DP44 sequences (SEQ ID NO: 32) and/or the V_{H} 3-23 sequences (SEQ ID NO:34) and/or the V_{H} 3-7 sequences (SEQ ID NO:35) and/or the Vₖ L6 framework sequence (SEQ ID NO:33) used by preferred monoclonal antibodies of the invention. The V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

Accordingly, in another embodiment, the invention provides isolated anti-IRTA-5 monoclonal antibodies, or antigen binding portions thereof, comprising a heavy chain variable region comprising: (a) a V_{H} CDR1 region comprising an amino acid sequence selected from the group consisting of SEQ ID NOs: 1, 2, and 3, or an amino acid sequence having one, two, three, four or five amino acid substitutions, deletions or additions as compared to SEQ ID NOs: 1, 2, and 3; (b) a V_{H} CDR2 region comprising an amino acid sequence selected from the group consisting of SEQ ID NOs: 4, 5, and 6, or an amino acid sequence having one, two, three, four or five amino acid substitutions, deletions or additions as compared to SEQ ID NOs: 4, 5, and 6; (c) a V_{H} CDR3 region comprising an amino acid sequence selected from the group consisting of SEQ ID NOs: 7, 8, and 9, or an amino acid sequence having one, two, three, four or five amino acid substitutions, deletions or additions as compared to SEQ ID NOs: 7, 8, and 9; (d) a V_{K} CDR1 region comprising an amino acid sequence selected from the group consisting of SEQ ID NOs: 10, 11, and 12, or an amino acid sequence having one, two, three, four or five amino acid substitutions, deletions or additions as compared to SEQ ID NOs: 10, 11, and 12; (e) a V_{K} CDR2 region comprising an amino acid sequence selected from the group consisting of SEQ ID NOs: 13, 14, and 15, or an amino acid sequence having one, two, three, four or five amino acid substitutions, deletions or additions as compared to SEQ ID NOs: 13, 14, and 15; and (f) a V_{K} CDR3 region comprising an amino acid sequence selected from the group consisting of SEQ ID NOs: 16, 17, and 18, or an amino acid sequence having one, two, three, four or five amino acid substitutions, deletions or additions as compared to SEQ ID NOs: 16, 17, and 18.

Engineered antibodies of the invention include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. For example, for 2G5, amino acid residue #4 (within FR1) of V_{H} is a valine whereas this residue in the corresponding V_{H} 3-33 germline sequence is a leucine. To return the framework region sequences to their germline configuration, the somatic mutations can be “backmutated” to the germline sequence by, for example, site-directed mutagenesis or PCR-mediated mutagenesis (e.g., residue #4 of FR1 of the V_{H} of 5A2 can be “backmutated” from valine to leucine).

As another example, for 7G8, amino acid residue #1 (within FR1) of V_{H} is an aspartic acid whereas this residue in the corresponding V_{H} DP44 germline sequence is a glutamic acid. To return the framework region sequences to their germline configuration, for example, residue #1 of the V_{H} of 7G8 can be “backmutated” from aspartic acid to glutamic acid. Such “backmutated” antibodies are also intended to be encompassed by the invention.

As yet another example, for 7G8, amino acid residue #3 (within FR1) of V_{H} is histidine whereas this residue in the corresponding V_{H} DP44 germline sequence is a glutamine. To return the framework region sequences to their germline configuration, for example, residue #3 of the V_{H} of 7G8 can be “backmutated” from histidine to glutamine. Such “backmutated” antibodies are also intended to be encompassed by the invention.

As yet another example, for 7G8, amino acid residue #37 (within FR2) of V_{H} is an isoleucine whereas this residue in the corresponding V_{H} DP44 germline sequence is a valine. To return the framework region sequences to their germline configuration, for example, residue #37 (residue #2 of FR2) of the V_{H} of 7G8 can be “backmutated” from isoleucine to valine. Such “backmutated” antibodies are also intended to be encompassed by the invention.

As yet another example, for 7G8, amino acid residue #44 (within FR2) of V_{H} is an aspartic acid whereas this residue in the corresponding V_{H} DP44 germline sequence is a glycine. To return the framework region sequences to their germline configuration, for example, residue #44 (residue #9 of FR2) of the V_{H} of 7G8 can be “backmutated” from aspartic acid to glycine. Such “backmutated” antibodies are also intended to be encompassed by the invention.

As yet another example, for 2G5, amino acid residue #85 (within FR3) of V_{K} is a leucine whereas this residue in the corresponding V_{K} L6 germline sequence is a valine. To return the framework region sequences to their germline configuration, for example, residue #85 (residue #29 of FR3) of the V_{K} of 2G5 can be “backmutated” from leucine to valine. Such “backmutated” antibodies are also intended to be encompassed by the invention.

In a preferred embodiment, certain residues within V_{H} of 7G8 are mutated to residues identical to or similar to residues in other human germline sequences (discussed further in Example 3). For example, the invention also provides a heavy chain variable region of 7G8(mut) in which the histidine at position 13 has been mutated to lysine or glutamine and the methionine at position 87 has been mutated to threonine. The amino acid sequence of the V_{H} of 7G8(mut) is shown in SEQ ID NO: 36. Accordingly, in another embodiment, the invention provides an antibody comprising a heavy chain variable region comprising the amino acid sequence of SEQ ID NO: 36 and a light chain variable region comprising the amino acid sequence of SEQ ID NOs: 22, 23 or 24, preferably SEQ ID NO: 24.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or C_{L} region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono(C1-C10)alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al and EP 0 401 384 by Ishikawa et al.

### Methods of Engineering Antibodies

As discussed above, the anti-IRTA-5 antibodies having V_{H} and V_{K} sequences disclosed herein can be used to create new anti-IRTA-5 antibodies by modifying the V_{H} and/or V_{K} sequences, or the constant region(s) attached thereto. Thus, in another aspect of the invention, the structural features of an anti-IRTA-5 antibody of the invention, e.g. 2G5, 5A2, or 7G8, are used to create structurally related anti-IRTA-5 antibodies that retain at least one functional property of the antibodies of the invention, such as binding to human IRTA-5. For example, one or more CDR regions of 2G5, 5A2, or 7G8, or mutations thereof, can be combined recombinantly with known framework regions and/or other CDRs to create additional, recombinantly-engineered, anti-IRTA-5 antibodies of the invention, as discussed above. Other types of modifications include those described in the previous section. The starting material for the engineering method is one or more of the V_{H} and/or V_{K} sequences provided herein, or one or more CDR regions thereof. To create the engineered antibody, it is not necessary to actually prepare (i.e., express as a protein) an antibody having one or more of the V_{H} and/or V_{K} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing an anti-IRTA-5 antibody comprising:
(a) providing: (i) a heavy chain variable region antibody sequence comprising a CDR1 sequence selected from the group consisting of SEQ ID NOs: 1, 2, and 3, a CDR2 sequence selected from the group consisting of SEQ ID NOs: 4, 5, and 6 and/or a CDR3 sequence selected from the group consisting of SEQ ID NOs: 7, 8, and 9; and/or (ii) a light chain variable region antibody sequence comprising a CDR1 sequence selected from the group consisting of SEQ ID NOs: 10, 11, and 12, a CDR2 sequence selected from the group consisting of SEQ ID NOs: 13, 14, and 15 and/or a CDR3 sequence selected from the group consisting of SEQ ID NOs: 16, 17, and 18;
(b) altering at least one amino acid residue within the heavy chain variable region antibody sequence and/or the light chain variable region antibody sequence to create at least one altered antibody sequence; and
(c) expressing the altered antibody sequence as a protein.

Standard molecular biology techniques can be used to prepare and express the altered antibody sequence.

Preferably, the antibody encoded by the altered antibody sequence(s) is one that retains one, some or all of the functional properties of the anti-IRTA-5 antibodies described herein, which functional properties include, but are not limited to:
(i) binds to human IRTA-5 with a K_{D} of 5×10⁻⁸ M or less;
(ii) does not substantially bind to human IRTA-1, IRTA-2, IRTA-3 and IRTA-4; and/or
(iii) binds to human B lymphocytes and to B cell tumor lines but does not substantially bind to CD3+ peripheral blood T cells, CD1A+ peripheral blood dendritic cells, CD14+ peripheral blood monocytes, or CD56+ peripheral blood natural killer cells.

The functional properties of the altered antibodies can be assessed using standard assays available in the art and/or described herein, such as those set forth in the Examples (e.g., flow cytometry, binding assays).

In certain embodiments of the methods of engineering antibodies of the invention, mutations can be introduced randomly or selectively along all or part of an anti-IRTA-5 antibody coding sequence and the resulting modified anti-IRTA-5 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Nucleic Acid Molecules Encoding Antibodies of the Invention

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Preferred nucleic acids molecules of the invention are those encoding the V_{H} and V_{L} sequences of the 2G5, 5A2, or 7G8 monoclonal antibodies. DNA sequences encoding the V_{H} sequences of 2G5, 5A2, and 7G8 are shown in SEQ ID NOs: 25, 26, and 27, respectively. DNA sequences encoding the V_{L} sequences of 2G5, 5A2, and 7G8 are shown in SEQ ID NOs: 28, 29, and 30, respectively.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the V_{H}— and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Production of Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against IRTA-5 can be generated using transgenic or transchromosomic mice carrying parts of the human immune system rather than the mouse system. These transgenic and transchromosomic mice include mice referred to herein as the HuMAb Mouse® and KM Mouse®, respectively, and are collectively referred to herein as “human Ig mice.”

The HuMAb Mouse® (Medarex®, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of the HuMab Mouse®, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication WO 02/43478 to Ishida et al.

Still further, alternative transgenic animal systems expressing human immunoglobulin genes are available in the art and can be used to raise anti-IRTA-5 antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-IRTA-5 antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-IRTA-5 antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Immunization of Human Ig Mice

When human Ig mice are used to raise human antibodies of the invention, such mice can be immunized with a purified or enriched preparation of IRTA-5 antigen and/or recombinant IRTA-5, or an IRTA-5 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of IRTA-5 antigen can be used to immunize the human Ig mice intraperitoneally.

Detailed procedures to generate fully human monoclonal antibodies to IRTA-5 are described in Example 1 below. Cumulative experience with various antigens has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-IRTA-5 human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo12). Alternatively or additionally, the KM Mouse® strain can be used.

### Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

### Generation of Transfectomas Producing Monoclonal Antibodies of the Invention

Antibodies of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to Antigen

Antibodies of the invention can be tested for binding to IRTA-5 by, for example, standard ELISA. Briefly, microtiter plates are coated with purified IRTA-5 at 0.25 μg/ml in PBS, and then blocked with 5% bovine serum albumin in PBS. Dilutions of antibody (e.g., dilutions of plasma from IRTA-5-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with IRTA-5 immunogen. Hybridomas that bind with high avidity to IRTA-5 are subcloned and further characterized. One clone from each hybridoma, which retains the reactivity of the parent cells (by ELISA), can be chosen for making a 5-10 vial cell bank stored at −140° C., and for antibody purification.

To purify anti-IRTA-5 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected anti-IRTA-5 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using IRTA-5 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-IRTA-5 human IgGs can be further tested for reactivity with IRTA-5 antigen by Western blotting. Briefly, IRTA-5 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Immunoconjugates

In another aspect, the present invention features an anti-IRTA-5 antibody, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg™; Wyeth).

Cytoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Method for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the antibodies of the invention.

The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### Bispecific Molecules

In another aspect, the present invention features bispecific molecules comprising an anti-IRTA-5 antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for IRTA-5 and a second binding specificity for a second target epitope. In a particular embodiment of the invention, the second target epitope is an Fc receptor, e.g., human FcγRI (CD64) or a human Fcα receptor (CD89). Therefore, the invention includes bispecific molecules capable of binding both to FcγR, FcαR or FcεR expressing effector cells (e.g., monocytes, macrophages or polymorphonuclear cells (PMNs)), and to target cells expressing IRTA-5. These bispecific molecules target IRTA-5 expressing cells to effector cell and trigger Fc receptor-mediated effector cell activities, such as phagocytosis of an IRTA-5 expressing cells, antibody dependent cell-mediated cytotoxicity (ADCC), cytokine release, or generation of superoxide anion.

In an embodiment of the invention in which the bispecific molecule is multispecific, the molecule can further include a third binding specificity, in addition to an anti-Fc binding specificity and an anti-IRTA-5 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one embodiment, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII (CD32), and FcγRIII (CD16). In one preferred embodiment, the Fcγ receptor a human high affinity FcγRI. The human FcγRI is a 72 kDa molecule, which shows high affinity for monomeric IgG (10⁸-10⁹ M⁻¹).

The production and characterization of certain preferred anti-Fcγ monoclonal antibodies are described by Fanger et al. in PCT Publication WO 88/00052 and in U.S. Pat. No. 4,954,617, the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al. (1995) J. Immunol. 155 (10): 4996-5002 and PCT Publication WO 94/10332. The H22 antibody producing cell line was deposited at the American Type Culture Collection under the designation HA022CL1 and has the accession no. CRL 11177.

In still other preferred embodiments, the binding specificity for an Fc receptor is provided by an antibody that binds to a human IgA receptor, e.g., an Fc-alpha receptor (FcαRI (CD89)), the binding of which is preferably not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 10 kDa. FcαRI (CD89) is constitutively expressed on monocytes/macrophages, eosinophilic and neutrophilic granulocytes, but not on non-effector cell populations. FcαRI has medium affinity (5×10⁷ M⁻¹) for both IgA1 and IgA2, which is increased upon exposure to cytokines such as G-CSF or GM-CSF (Morton, H. C. et al (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al (1992) J. Immunol. 148:1764).

FcαRI and FcγRI are preferred trigger receptors for use in the bispecific molecules of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules of the invention are murine, chimeric and humanized monoclonal antibodies.

The bispecific molecules of the present invention can be prepared by conjugating the constituent binding specificities, e.g., the anti-FcR and anti-IRTA-5 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### Pharmaceutical Compositions

In another aspect, the present invention provides a composition, e.g., a pharmaceutical composition, containing one or a combination of monoclonal antibodies, or antigen-binding portion(s) thereof, of the present invention, formulated together with a pharmaceutically acceptable carrier. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecifics) that bind to different epitopes on the target antigen or that have complementary activities.

Pharmaceutical compositions of the invention also can be administered in combination therapy, i.e., combined with other agents. For example, the combination therapy can include an anti-IRTA-5 antibody of the present invention combined with at least one other anti-inflammatory or immunosuppressant agent. Examples of therapeutic agents that can be used in combination therapy are described in greater detail below in the section on uses of the antibodies of the invention.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjuage, or bispecific molecule, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-IRTA-5 antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-IRTA-5 antibody of the invention preferably results in a decrease in severity of disease symptoms, an increase in frequency and duration of disease symptom-free periods, or a prevention of impairment or disability due to the disease affliction. For example, for the treatment of IRTA-5+tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al, (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### Uses and Methods of the Invention

The antibodies, particularly the human antibodies, antibody compositions and methods of the present invention have numerous in vitro and in vivo diagnostic and therapeutic utilities involving the diagnosis and treatment of IRTA-5 mediated disorders. For example, these molecules can be administered to cells in culture, in vitro or ex vivo, or to human subjects, e.g., in vivo, to treat, prevent and to diagnose a variety of disorders. As used herein, the term “subject” is intended to include human and non-human animals. Non-human animals includes all vertebrates, e.g., mammals and non-mammals, such as non-human primates, sheep, dogs, cats, cows, horses, chickens, amphibians, and reptiles. Preferred subjects include human patients having disorders mediated by IRTA-5 activity. The methods are particularly suitable for treating human patients having a disorder associated with aberrant IRTA-5 expression. When antibodies to IRTA-5 are administered together with another agent, the two can be administered in either order or simultaneously.

Given the specific binding of the antibodies of the invention for IRTA-5, compared to IRTA-1, 2, 3 and 4, the antibodies of the invention can be used to specifically detect IRTA-5 expression on the surface of cells and, moreover, can be used to purify IRTA-5 via immunoaffinity purification.

Furthermore, given the expression of IRTA-5 on various tumor cells, the human antibodies, antibody compositions and methods of the present invention can be used to treat a subject with a tumorigenic disorder, e.g., a disorder characterized by the presence of tumor cells expressing IRTA-5 including, for example, Burkitt's lymphoma, anaplastic large-cell lymphomas (ALC_{L}), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other B-cell lymphomas.

In one embodiment, the antibodies (e.g., human monoclonal antibodies, multispecific and bispecific molecules and compositions) of the invention can be used to detect levels of IRTA-5, or levels of cells which contain IRTA-5 on their membrane surface, which levels can then be linked to certain disease symptoms. Alternatively, the antibodies can be used to inhibit or block IRTA-5 function which, in turn, can be linked to the prevention or amelioration of certain disease symptoms, thereby implicating IRTA-5 as a mediator of the disease. This can be achieved by contacting a sample and a control sample with the anti-IRTA-5 antibody under conditions that allow for the formation of a complex between the antibody and IRTA-5. Any complexes formed between the antibody and IRTA-5 are detected and compared in the sample and the control.

In another embodiment, the antibodies (e.g., human antibodies, multispecific and bispecific molecules and compositions) of the invention can be initially tested for binding activity associated with therapeutic or diagnostic use in vitro. For example, compositions of the invention can be tested using the flow cytometric assays described in the Examples below.

The antibodies (e.g., human antibodies, multispecific and bispecific molecules, immunoconjugates and compositions) of the invention have additional utility in therapy and diagnosis of IRTA-5-related diseases. For example, the human monoclonal antibodies, the multispecific or bispecific molecules and the immunoconjugates can be used to elicit in vivo or in vitro one or more of the following biological activities: to inhibit the growth of and/or kill a cell expressing IRTA-5; to mediate phagocytosis or ADCC of a cell expressing IRTA-5 in the presence of human effector cells, or to block IRTA-5 ligand binding to IRTA-5.

In a particular embodiment, the antibodies (e.g., human antibodies, multispecific and bispecific molecules and compositions) are used in vivo to treat, prevent or diagnose a variety of IRTA-5-related diseases. Examples of IRTA-5-related diseases include, among others, cancer, non-Hodgkin's lymphoma, Burkitt's lymphoma, anaplastic large-cell lymphomas (ALC_{L}), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other B-cell lymphomas.

Suitable routes of administering the antibody compositions (e.g., human monoclonal antibodies, multispecific and bispecific molecules and immunoconjugates) of the invention in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, human anti-IRTA-5 antibodies of the invention can be co-administered with one or other more therapeutic agents, e.g., an cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the human anti-IRTA-5 antibodies, or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

Target-specific effector cells, e.g., effector cells linked to compositions (e.g., human antibodies, multispecific and bispecific molecules) of the invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing IRTA-5, and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) of the invention and/or effector cells armed with these compositions can be used in conjunction with chemotherapy. Additionally, combination immunotherapy may be used to direct two distinct cytotoxic effector populations toward tumor cell rejection. For example, anti-IRTA-5 antibodies linked to anti-Fc-gamma RI or anti-CD3 may be used in conjunction with IgG- or IgA-receptor specific binding agents.

Bispecific and multispecific molecules of the invention can also be used to modulate FcγR or FcγR levels on effector cells, such as by capping and elimination of receptors on the cell surface. Mixtures of anti-Fc receptors can also be used for this purpose.

The compositions (e.g., human antibodies, multispecific and bispecific molecules and immunoconjugates) of the invention which have complement binding sites, such as portions from IgG1, -2, or -3 or IgM which bind complement, can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

The compositions (e.g., human antibodies, multispecific and bispecific molecules and immunoconjugates) of the invention can also be administered together with complement. Accordingly, within the scope of the invention are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of the invention and the complement or serum can be administered separately.

Also within the scope of the present invention are kits comprising the antibody compositions of the invention (e.g., human antibodies, bispecific or multispecific molecules, or immunoconjugates) and instructions for use. The kit can further contain one or more additional reagents, such as an immunosuppressive reagent, a cytotoxic agent or a radiotoxic agent, or one or more additional human antibodies of the invention (e.g., a human antibody having a complementary activity which binds to an epitope in the IRTA-5 antigen distinct from the first human antibody).

Accordingly, patients treated with antibody compositions of the invention can be additionally administered (prior to, simultaneously with, or following administration of a human antibody of the invention) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNF).

The compositions (e.g., human antibodies, multispecific and bispecific molecules) of the invention can also be used to target cells expressing FcγR or IRTA-5, for example for labeling such cells. For such use, the binding agent can be linked to a molecule that can be detected. Thus, the invention provides methods for localizing ex vivo or in vitro cells expressing Fc receptors, such as FcγR, or IRTA-5. The detectable label can be, e.g., a radioisotope, a fluorescent compound, an enzyme, or an enzyme co-factor.

In a particular embodiment, the invention provides methods for detecting the presence of IRTA-5 antigen in a sample, or measuring the amount of IRTA-5 antigen, comprising contacting the sample, and a control sample, with a human monoclonal antibody, or an antigen binding portion thereof, which specifically binds to IRTA-5, under conditions that allow for formation of a complex between the antibody or portion thereof and IRTA-5. The formation of a complex is then detected, wherein a difference complex formation between the sample compared to the control sample is indicative the presence of IRTA-5 antigen in the sample.

In other embodiments, the invention provides methods for treating an IRTA-5 mediated disorder in a subject, e.g., cancer, non-Hodgkin's lymphoma, Burkitt's lymphoma, anaplastic large-cell lymphomas (ALC_{L}), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other B-cell lymphomas, by administering to the subject the human antibodies described above. Such antibodies and derivatives thereof are used to inhibit IRTA-5 induced activities associated with certain disorders, e.g., proliferation and differentiation. By contacting the antibody with IRTA-5 (e.g., by administering the antibody to a subject), the ability of IRTA-5 to induce such activities is inhibited and, thus, the associated disorder is treated. The antibody composition can be administered alone or along with another therapeutic agent, such as a cytotoxic or a radiotoxic agent which acts in conjunction with or synergistically with the antibody composition to treat or prevent the IRTA-5 mediated disease.

In yet another embodiment, immunoconjugates of the invention can be used to target compounds (e.g., therapeutic agents, labels, cytotoxins, radiotoxoins immunosuppressants, etc.) to cells which have IRTA-5 cell surface receptors by linking such compounds to the antibody. Thus, the invention also provides methods for localizing ex vivo or in vivo cells expressing IRTA-5 (e.g., with a detectable label, such as a radioisotope, a fluorescent compound, an enzyme, or an enzyme co-factor). Alternatively, the immunoconjugates can be used to kill cells which have IRTA-5 cell surface receptors by targeting cytotoxins or radiotoxins to IRTA-5.

The present invention is further illustrated by the following examples which should not be construed as further limiting. The contents of all figures and all references, patents and published patent applications cited throughout this application are expressly incorporated herein by reference.

### EXAMPLES

### Example 1

### Generation of Human Monoclonal Antibodies Against IRTA5

### Antigen

A fusion protein composed of the extracellular domain of the IRTA5 linked to a heterologous polypeptide was generated by standard recombinant methods and used as antigen for immunization.

### Transgenic HuMab Mouse®

Fully human monoclonal antibodies to IRTA5 were prepared using mice from the HCo7 strain of the transgenic HuMab Mouse®, which expresses human antibody genes. In this mouse strain, the endogenous mouse kappa light chain gene has been homozygously disrupted as described in Chen et al (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al (1996) Nature Biotechnology 14:845-851, and a human heavy chain transgene, HCo7, as described in U.S. Pat. Nos. 5,545,806; 5,625,825; and 5,545,807.

### HuMab Immunizations:

To generate fully human monoclonal antibodies to IRTA5, mice of the HCo7 HuMab Mouse® strain were immunized with purified recombinant IRTA5 fusion protein derived from mammalian cells that had been transfected with an expression vector containing the gene encoding the fusion protein. General immunization schemes for the HuMab Mouse® are described in Lonberg, N. et al (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. A purified recombinant IRTA5 antigen preparation (5-50 μg, purified from transfected mammalian cells expressing IRTA5 fusion protein) was used to immunize the HuMab mice Intraperitoneallymice™ intraperitonealy (IP).

Transgenic mice were immunized twice with antigen in complete Freund's adjuvant or Ribi adjuvant IP, followed by 3-21 days IP (up to a total of 11 immunizations) with the antigen in incomplete Freund's or Ribi adjuvant. The immune response was monitored by retroorbital bleeds. The plasma was screened by ELISA (as described below), and mice with sufficient titers of anti-IRTA5 human immunoglobulin were used for fusions. Mice were boosted intravenously with antigen 3 days before sacrifice and removal of the spleen.

### Selection of HuMab Mice™ Producing Anti-IRTA5 Antibodies:

To select HuMab Mice™ producing antibodies that bound IRTA5, sera from immunized mice was tested by a modified ELISA as originally described by Fishwild, D. et al. (1996). Briefly, microtiter plates were coated with purified recombinant IRTA5 fusion protein at 1-2 μg/ml in PBS, 50 μl/wells incubated 4° C. overnight then blocked with 200 μl/well of 5% BSA in PBS. Dilutions of plasma from IRTA5-immunized mice were added to each well and incubated for 1-2 hours at ambient temperature. The plates were washed with PBS/Tween and then incubated with a goat-anti-human kappa light chain polyclonal antibody conjugated with alkaline phosphatase for 1 hour at room temperature. After washing, the plates were developed with pNPP substrate and analyzed by spectrophotometer at OD 415-650. Mice that developed the highest titers of anti-IRTA5 antibodies were used for fusions. Fusions were performed as described below and hybridoma supernatants were tested for anti-IRTA5 activity by ELISA.

### Generation of Hybridomas Producing Human Monoclonal Antibodies to IRTA5:

The mouse splenocytes, isolated from the HuMab Mice™, were fused with PEG to a mouse myeloma cell line based upon standard protocols. The resulting hybridomas were then screened for the production of antigen-specific antibodies. Single cell suspensions of splenic lymphocytes from immunized mice were fused to one-fourth the number of P3X63 Ag8.6.53 (ATCC CRL 1580) nonsecreting mouse myeloma cells with 50% PEG (Sigma). Cells were plated at approximately 1×10⁵/well in flat bottom microtiter plate, followed by about two week incubation in selective medium containing 10% fetal calf serum, supplemented with origen (IGEN) in RPMI, L-glutamine, sodium pyruvate, HEPES, penicillin, streptamycin, gentamycin, 1×HAT, and beta-mercaptoethanol. After 1-2 weeks, cells were cultured in medium in which the HAT was replaced with HT. Individual wells were then screened by ELISA (described above) for human anti-IRTA5 monoclonal IgG antibodies. Once extensive hybridoma growth occurred, medium was monitored usually after 10-14 days. The antibody secreting hybridomas were replated, screened again and, if still positive for human IgG, anti-IRTA5 monoclonal antibodies were subcloned at least twice by limiting dilution. The stable subclones were then cultured in vitro to generate small amounts of antibody in tissue culture medium for further characterization.

Hybridoma clones 2G2, 2G5, 5A2, 7G8, 1E5, 4B7, and 7F5 were selected for further analysis.

### Example 2

### Structural Characterization of Human Monoclonal Antibodies 5A2, 2G5 and 7G8

The cDNA sequences encoding the heavy and light chain variable regions of the 2G5, 5A2, and 7G8 monoclonal antibodies were obtained from the 2G5, 5A2, and 7G8 hybridomas, respectively, using standard PCR techniques and were sequenced using standard DNA sequencing techniques.

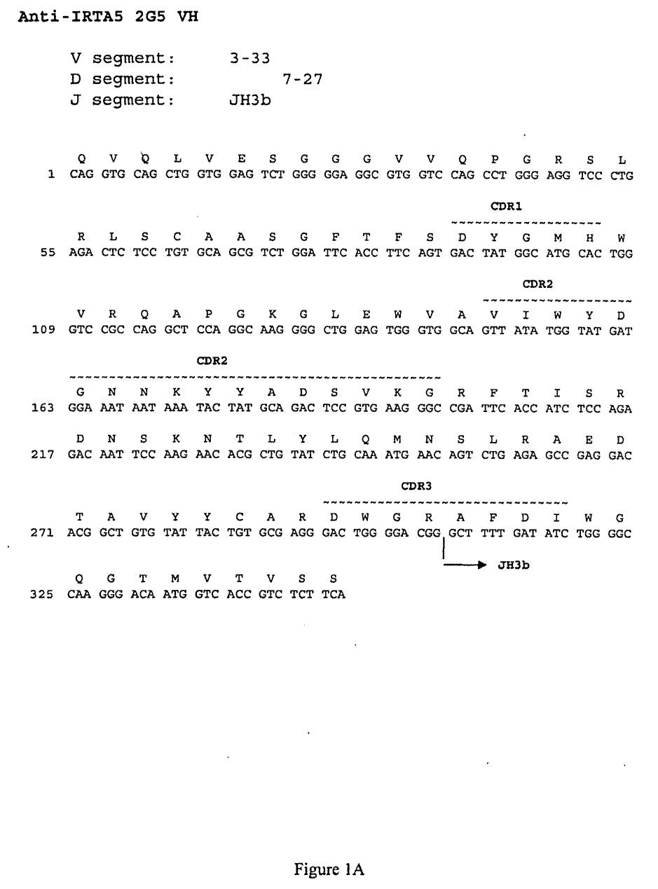

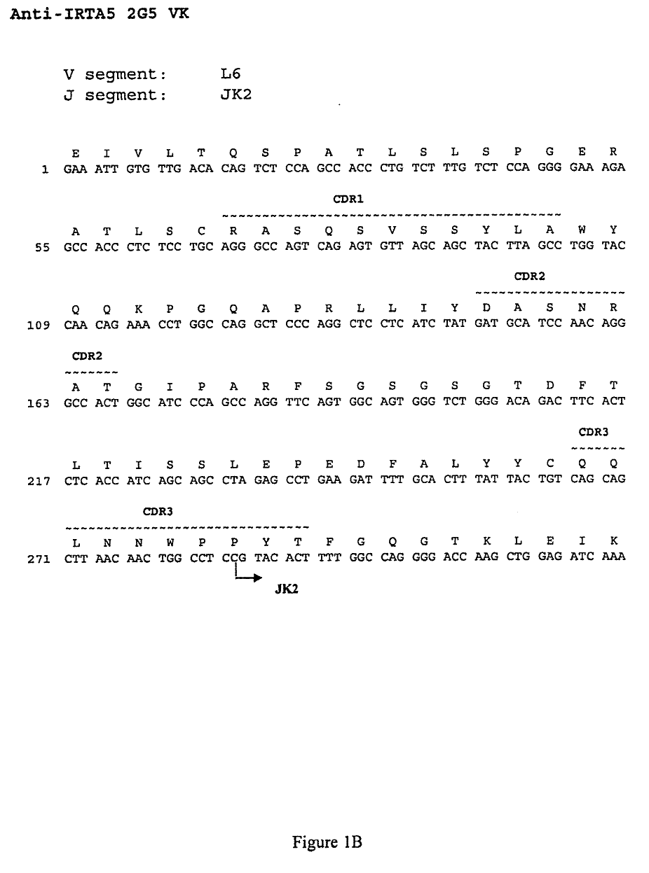

The nucleotide and amino acid sequences of the heavy chain variable region of 2G5 are shown in FIG. 1A and in SEQ ID NO: 25 and 19, respectively.

The nucleotide and amino acid sequences of the light chain variable region of 2G5 are shown in FIG. 1B and in SEQ ID NO: 28 and 22, respectively.

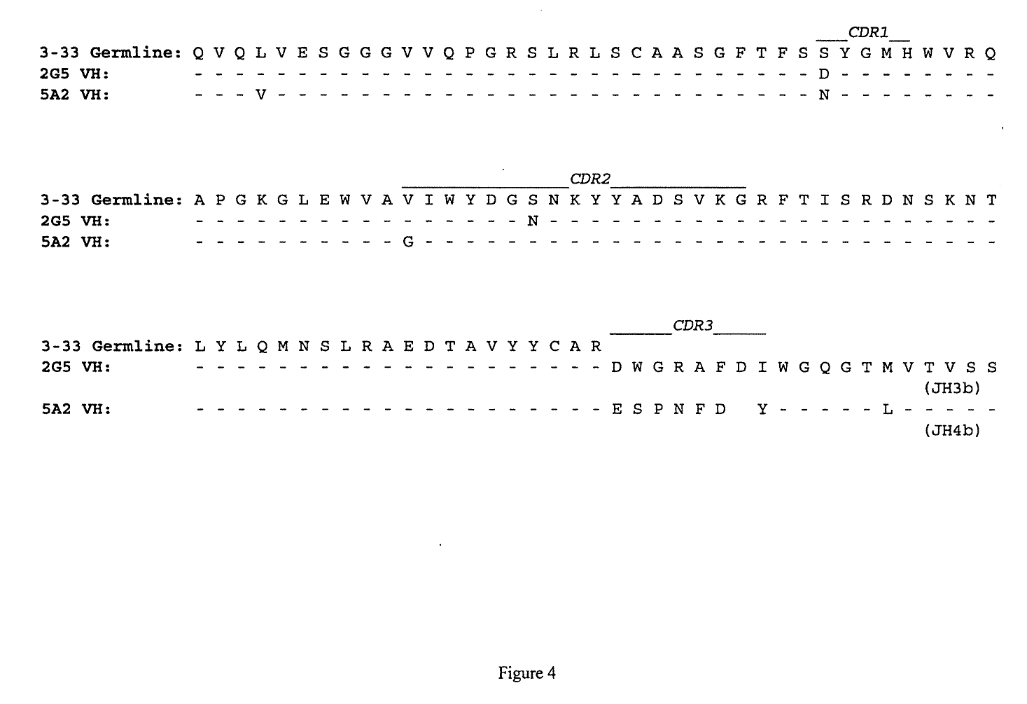

Comparison of the 2G5 heavy chain immunoglobulin sequence to the known human germline immunoglobulin heavy chain sequences demonstrated that the 2G5 heavy chain utilizes a V_{H} segment from human germline V_{H} 3-33, a D segment from the human germline 7-27, and a JH segment from human germline JH 3b. The alignment of the 2G5 V_{H} sequence to the germline V_{H} 3-33 sequence is shown in FIG. 4. Further analysis of the 2G5 V_{H} sequence using the Kabat system of CDR region determination led to the delineation of the heavy chain CDR1, CDR2 and CD3 regions as shown in FIGS. 1A and 4, and in SEQ ID NOs: 1, 4 and 7, respectively.

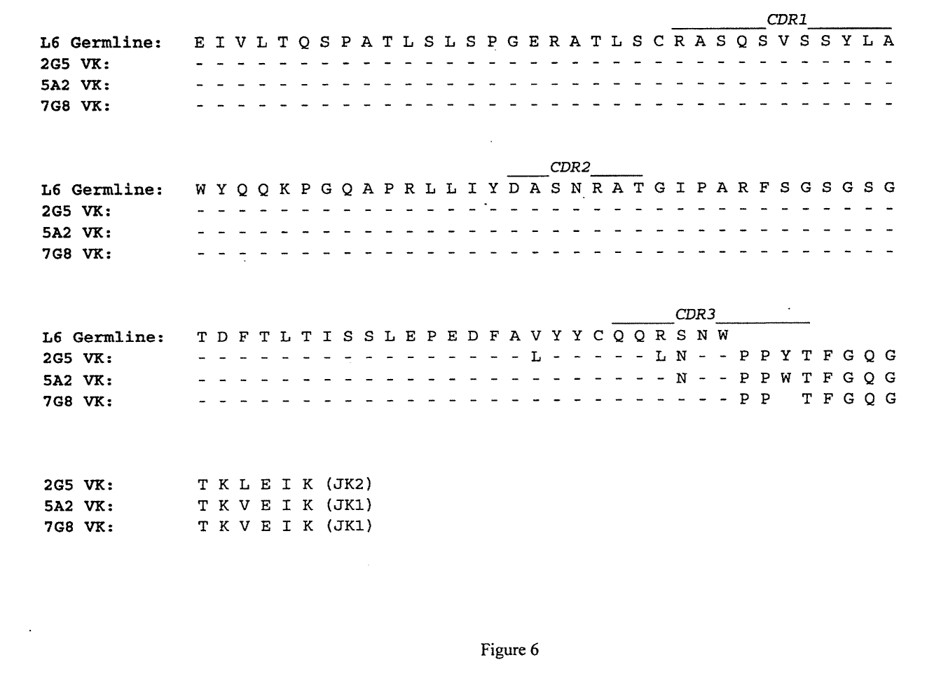

Comparison of the 2G5 light chain immunoglobulin sequence to the known human germline immunoglobulin light chain sequences demonstrated that the 2G5 light chain utilizes a V_{L} segment from human germline V_{K} L6 and a JK segment from human germline JK 2. The alignment of the 2G5 V_{L} sequence to the germline V_{K} L6 sequence is shown in FIG. 6. Further analysis of the 2G5 V_{L} sequence using the Kabat system of CDR region determination led to the delineation of the light chain CDR1, CDR2 and CD3 regions as shown in FIGS. 1B and 6, and in SEQ ID NOs:10, 13 and 16, respectively.

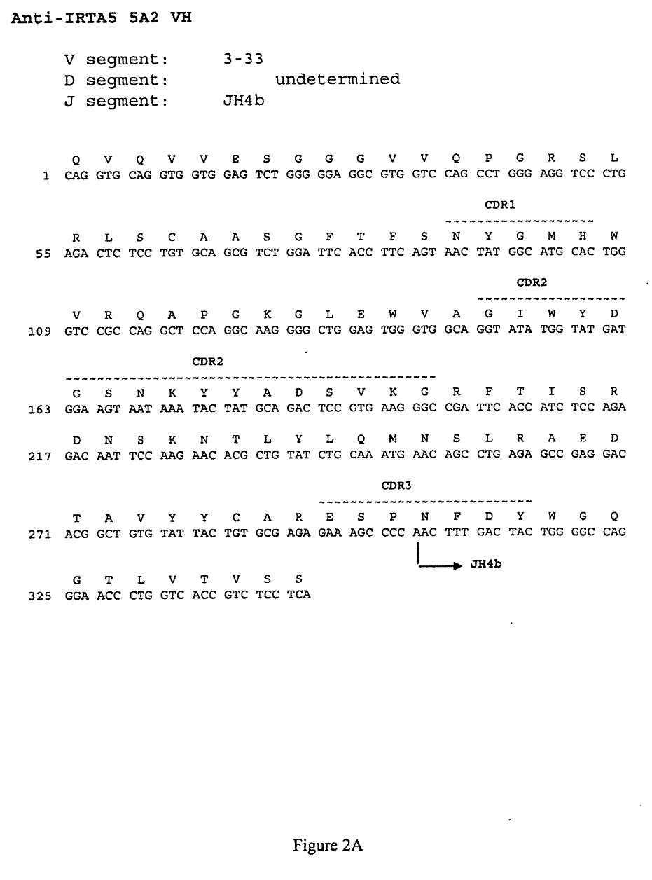

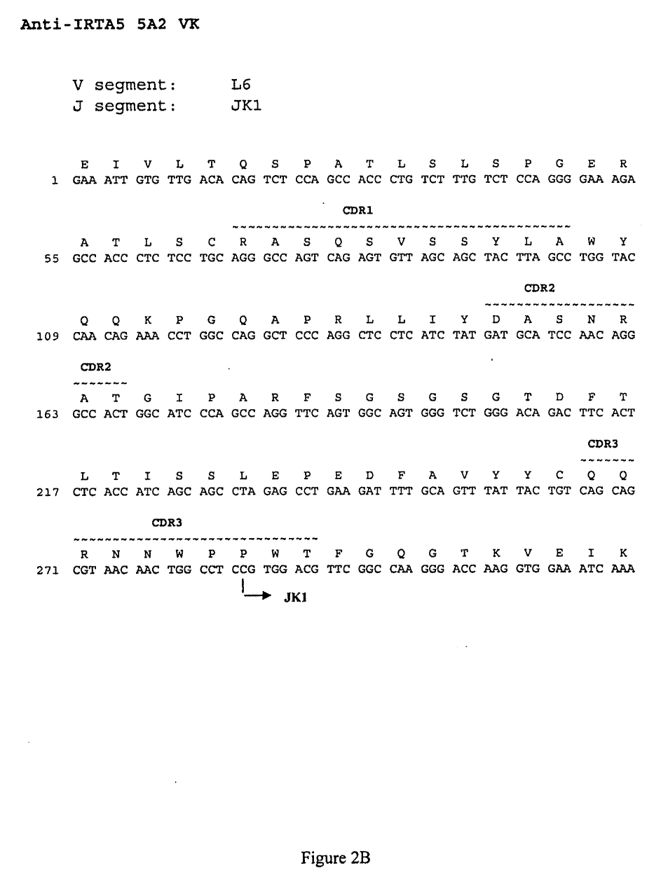

The nucleotide and amino acid sequences of the heavy chain variable region of 5A2 are shown in FIG. 2A and in SEQ ID NO: 26 and 20, respectively.

The nucleotide and amino acid sequences of the light chain variable region of 5A2 are shown in FIG. 2B and in SEQ ID NO: 29 and 23, respectively.

Comparison of the 5A2 heavy chain immunoglobulin sequence to the known human germline immunoglobulin heavy chain sequences demonstrated that the 5A2 heavy chain utilizes a V_{H} segment from human germline V_{H} 3-33, an undetermined D segment, and a JH segment from human germline JH 4b. The alignment of the 5A2 V_{H} sequence to the germline V_{H} 3-33 sequence is shown in FIG. 4. Further analysis of the 5A2 V_{H} sequence using the Kabat system of CDR region determination led to the delineation of the heavy chain CDR1, CDR2 and CD3 regions as shown in FIGS. 2A and 4, and in SEQ ID NOs: 2, 5 and 8, respectively.

Comparison of the 5A2 light chain immunoglobulin sequence to the known human germline immunoglobulin light chain sequences demonstrated that the 5A2 light chain utilizes a V_{L} segment from human germline V_{K} L6 and a JK segment from human germline JK 1. The alignment of the 5A2 V_{L} sequence to the germline V_{K} L6 sequence is shown in FIG. 6. Further analysis of the 5A2 V_{L} sequence using the Kabat system of CDR region determination led to the delineation of the light chain CDR1, CDR2 and CD3 regions as shown in FIGS. 2B and 6, and in SEQ ID NOs:11, 14 and 17, respectively.

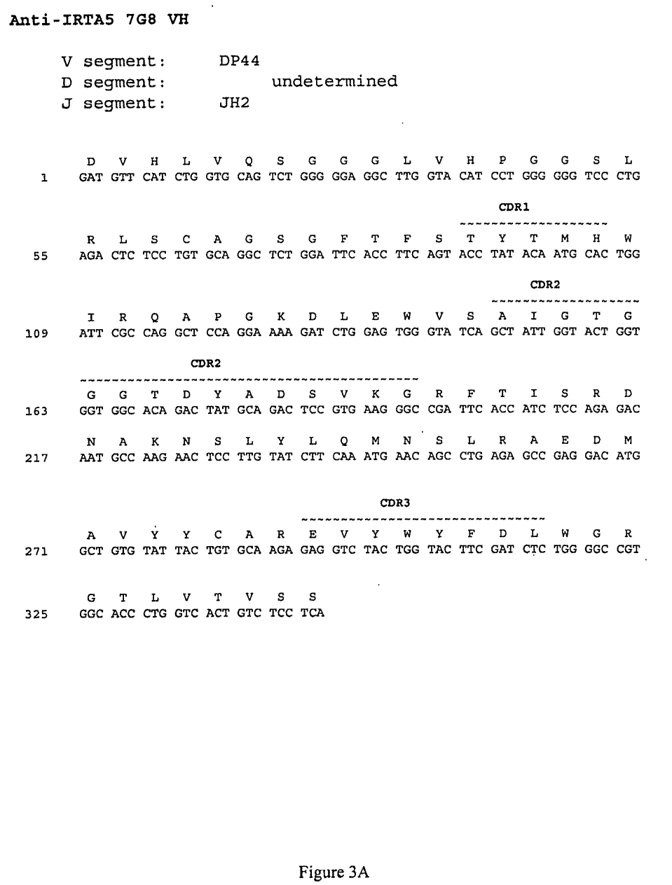

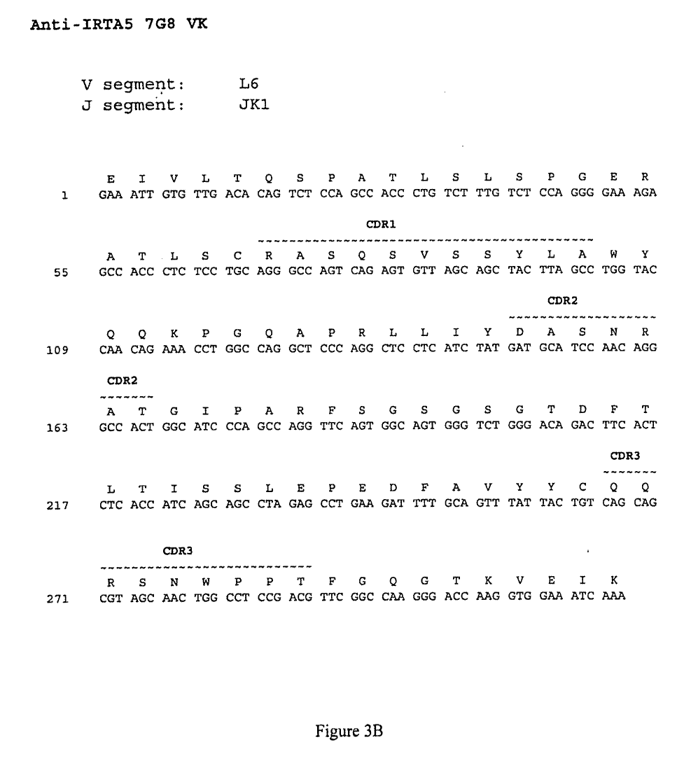

The nucleotide and amino acid sequences of the heavy chain variable region of 7G8 are shown in FIG. 3A and in SEQ ID NO: 27 and 21, respectively.

The nucleotide and amino acid sequences of the light chain variable region of 7G8 are shown in FIG. 3B and in SEQ ID NO: 30 and 24, respectively.

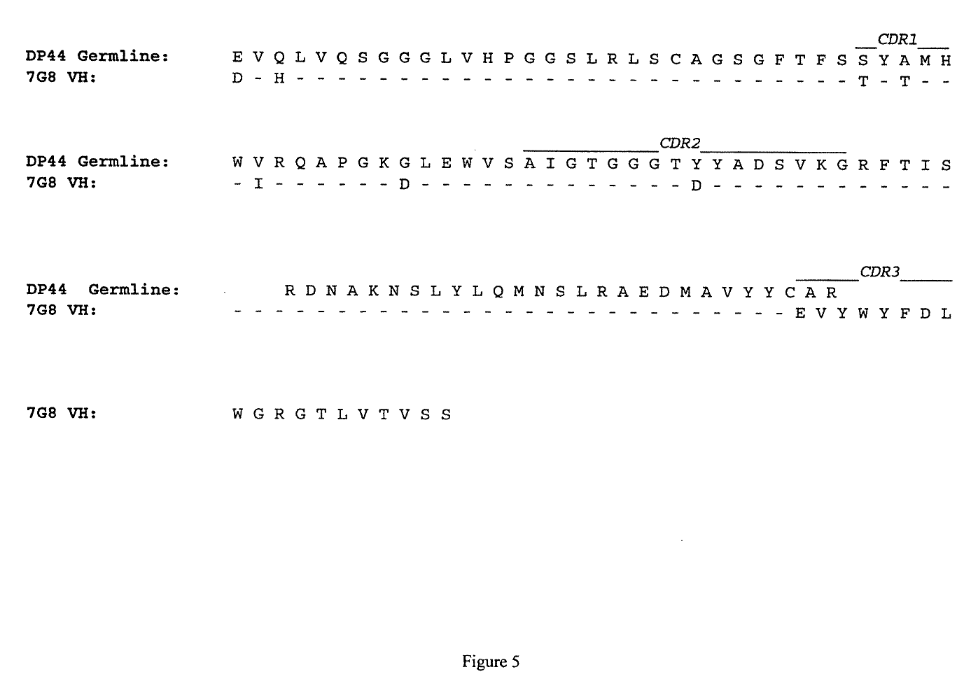

Comparison of the 7G8 heavy chain immunoglobulin sequence to the known human germline immunoglobulin heavy chain sequences demonstrated that the 7G8 heavy chain utilizes a V_{H} segment from human germline V_{H} DP44, an undetermined D segment, and a JH segment from human germline JH 2. The alignment of the 7G8 V_{H} sequence to the germline V_{H} DP44 sequence is shown in FIG. 5. Further analysis of the 7G8 V_{H} sequence using the Kabat system of CDR region determination led to the delineation of the heavy chain CDR1, CDR2 and CD3 regions as shown in FIGS. 3A and 5, and in SEQ ID NOs: 3, 6 and 9, respectively.

Comparison of the 7G8 light chain immunoglobulin sequence to the known human germline immunoglobulin light chain sequences demonstrated that the 7G8 light chain utilizes a V_{L} segment from human germline V_{K} L6 and a JK segment from human germline JK 1. The alignment of the 7G8 V_{L} sequence to the germline V_{K} A27 sequence is shown in FIG. 6. Further analysis of the 7G8 V_{L} sequence using the Kabat system of CDR region determination led to the delineation of the light chain CDR1, CDR2 and CD3 regions as shown in FIGS. 3B and 6, and in SEQ ID NOs:12, 15 and 18, respectively.

### Example 3

### Mutation of mAb 7G8 and Alternative Germline Usage

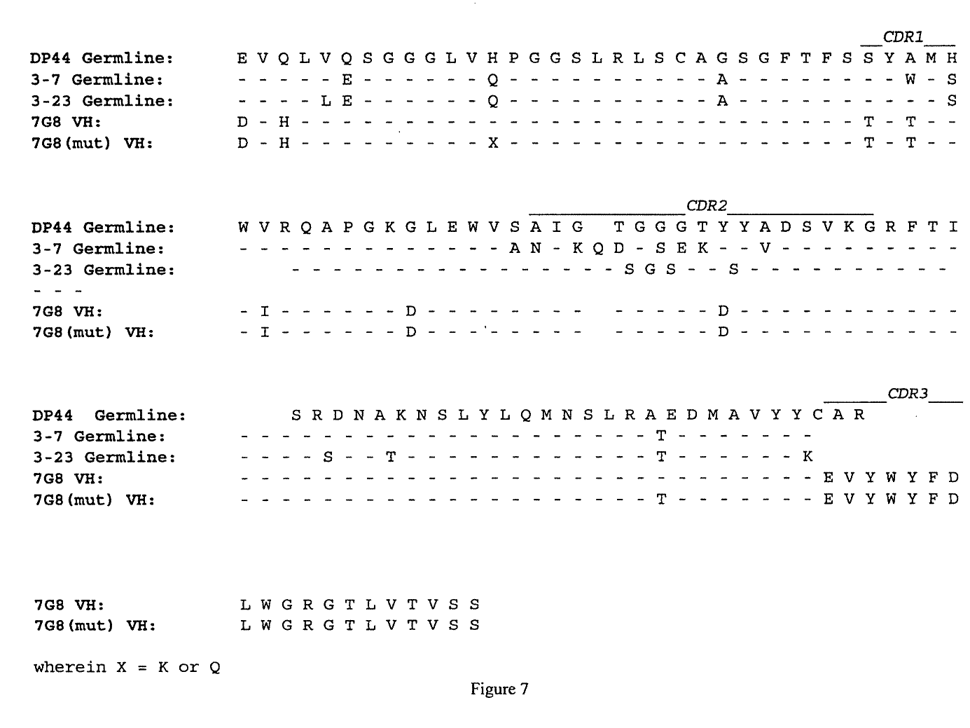

As discussed in Example 2 above, mAb 7G8 utilizes a heavy chain variable region derived from a human DP-44 germline sequence present in the HCo7 transgene of the HuMab Mouse® strain. Since DP-44 is not a germline sequence that is utilized in the native human immunoglobulin repertoire, it may be advantageous to mutate the V_{H} sequence of 7G8 to reduce potential immunogenicity. Preferably, one or more framework residues of the 7G8 V_{H} sequence is mutated to a residue(s) present in the framework of a structurally related V_{H} germline sequence that is utilized in the native human immunoglobulin repertoire. For example, FIG. 7 shows the alignment of the 7G8 V_{H} sequence with the DP44 germline sequence and also to two structurally related human germline sequences, V_{H} 3-23 and V_{H} 3-7. Given the relatedness of these sequences, one can predict that one can select a human antibody that specifically binds to human IRTA5 and that utilizes a V_{H} region derived from a V_{H} 3-23 or V_{H} 3-7. Moreover, one can mutate one or more residues within the 7G8 V_{H} sequence that differ from the residue(s) at the comparable position in the V_{H} 3-23 or V_{H} 3-7 sequence to the residue(s) that is present in V_{H} 3-23 or V_{H} 3-7, or to a conservative amino acid substitution thereof. For example, a preferred mutated form of 7G8 provided herein is referred to as 7G8(mut) and has the amino acid sequence shown in FIG. 7 and in SEQ ID NO: 36. In 7G8(mut), the histidine at amino acid position 13 has been mutated to either lysine or glutamine and the methionine at position 87 has been mutated to threonine.

### Example 4

### Characterization of Binding Specificity and Binding Kinetics of Anti-IRTA5 Human Monoclonal Antibodies

In this example, binding affinity, binding kinetics, binding specificity, and cross-competition of anti-IRTA5 antibodies were examined by Biacore analysis. Also, binding specificity was examined by flow cytometry.

### Binding Affinity and Kinetics

Anti-IRTA5 antibodies were characterized for affinities and binding kinetics by Biacore analysis (Biacore AB, Uppsala, Sweden). Purified recombinant human IRTA5 fusion protein was covalently linked to a CM5 chip (carboxy methyl dextran coated chip) via primary amines, using standard amine coupling chemistry and kit provided by Biacore. Binding was measured by flowing the antibodies in HBS EP buffer (provided by Biacore AB) at a concentration of 267 nM at a flow rate of 50 μl/min. The antigen-antibody association kinetics was followed for 3 minutes and the dissociation kinetics was followed for 7 minutes. The association and dissociation curves were fit to a 1:1 Langmuir binding model using BIAevaluation software (Biacore AB). To minimize the effects of avidity in the estimation of the binding constants, only the initial segment of data corresponding to association and dissociation phases were used for fitting. The K_{D}, kₒₙ and k_{off} values that were determined are shown in Table 1.

TABLE 1
Biacore binding data for IRTA5 HuMAbs.

| Sample | Sample | Affinity K_{D} × | On rate kₒₙ × | Off rate k_{off} × |
|---|---|---|---|---|
| # | ID | 10⁻⁹ (M) | 10⁵ (l/Ms) | 10⁻⁴ l/s |
| 1 | 2G5 | 0.028 | 1.52 | 0.043 |
| 2 | 5A2 | 0.035 | 2.52 | 0.087 |
| 3 | 7G8 | 16.8 | 0.72 | 12.1 |
| 4 | 1E5 | 17.1 | 0.23 | 3.93 |
| 5 | 7F5 | 19.3 | 0.72 | 13.9 |
| 6 | 4B7 | 25.4 | 0.46 | 11.8 |
| 7 | 2G1 | 42.3 | 0.31 | 13.3 |

### Epitope Mapping of Anti-IRTA5 Antibodies

Biacore was used to determine epitope grouping of anti-IRTA5 HuMAbs. Anti-IRTA5 antibodies (2G5, 5A2, 7G8, 4B7, 7F5, 4B7, 2G1) were used to map their epitopes on IRTAS. Antibodies 2G5, 5A2, and 7G8 were coated on three different surfaces of the same chip to 8000 RUs each. Dilutions of each of the above 7 mAbs were made, starting at 10 μg/mL and was incubated with IRTA5-Fc (50 nM) for one hour. The incubated complex was injected over all the three surfaces (and a blank surface) at the same time for 1.5 minutes at a flow rate of 20 μL/min. Signal from each surface at end of 1.5 minutes, after subtraction of appropriate blanks, has been plotted against concentration of mAb in the complex. Upon analysis of the data, the seven anti-IRTA5 antibodies have been categorized into 3 epitope groups group A, which includes 2G5, 5A2, and 7G8, group B1, which includes 7G8 and 1E5, and group B2, which includes 7F5, 4B7 and 2G1. The inter-relationship of the three epitope groups is illustrated schematically in FIG. 8.

### Binding Specificity by Flow Cytometry

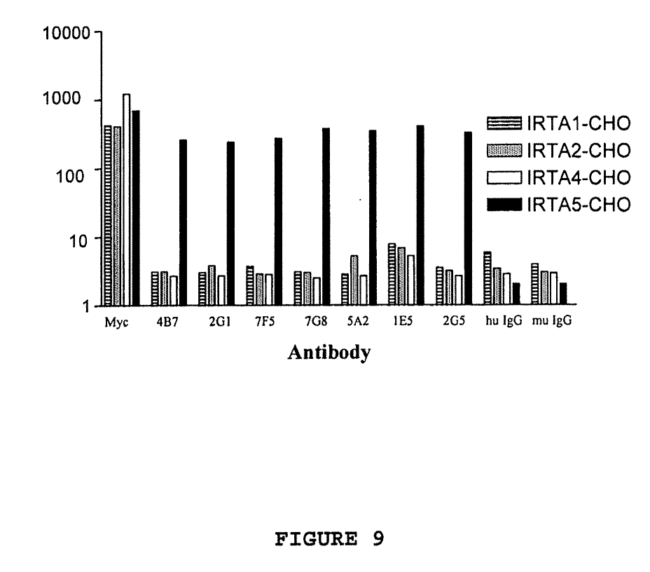

Chinese hamster ovary (CHO) cell lines that express one of each of the five IRTA proteins at the cell surface were developed and used to determine the specificity of the IRTA5 HuMAbs by flow cytometry. CHO cells were transfected with expression plasmids containing full length cDNA encoding transmembrane forms of IRTA1, IRTA2, IRTA3, IRTA4, or IRTA5. In addition, the transfected proteins contained an epitope tag at the N-terminus for detection by an antibody specific for the epitope. Binding of the seven anti-IRTA5 HuMAbs was assessed by incubating the transfected cells with each of the IRTA5 Abs at a concentration of 10 μg/ml. The cells were washed and binding was detected with a FITC-labeled anti-human IgG Ab. A murine anti-epitope tag Ab, followed by labeled anti-murine IgG, was used as the positive control. Non-specific human and murine Abs were used as negative controls. The results are depicted in FIG. 9. The IRTA5 HuMAbs bound to the CHO line transfected with IRTA5 but not to CHO lines expressing IRTA1, 2, or 4 as measured by the mean fluorescent intensity (MFI) of staining. Subsequently the HuMAbs were shown to have no specific binding to a CHO line expressing IRTA3 (data not shown). These data demonstrate the specificity of the HuMAbs for IRTA5.

### Example 5

### Binding of the IRTA5 Antibodies to Normal B Cells and to B Cell-Derived Tumor Lines

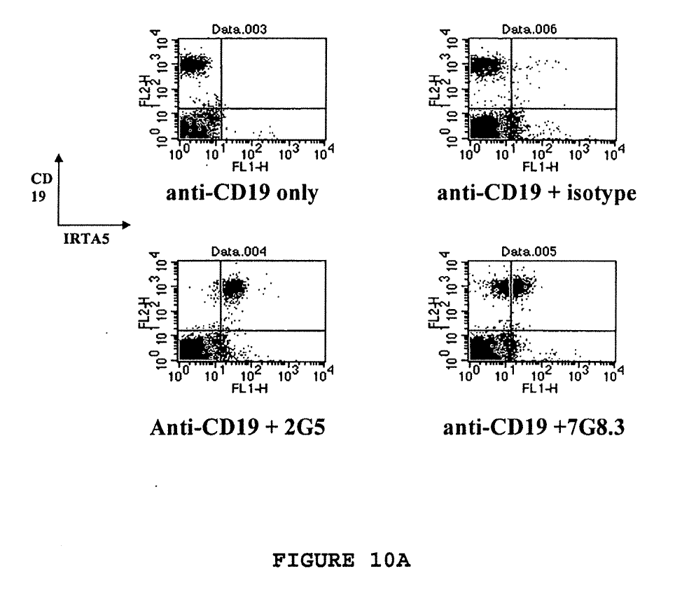

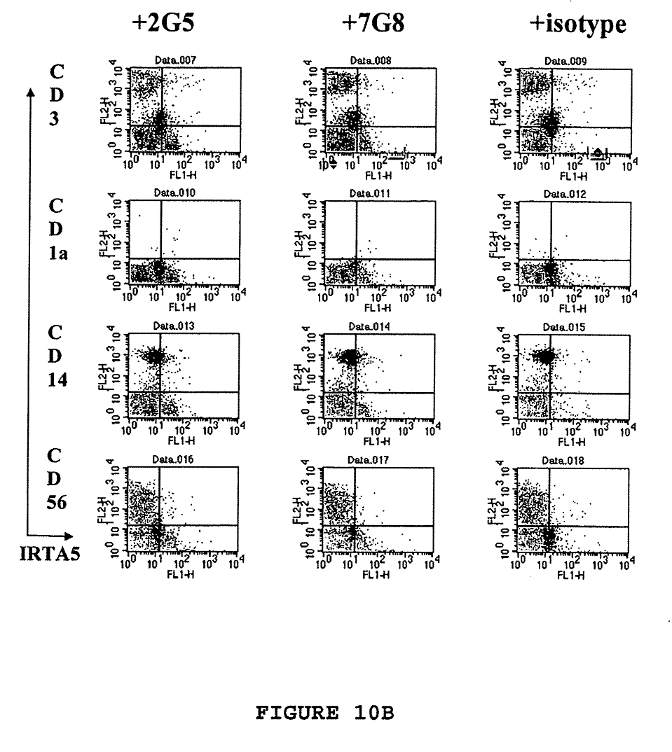

Two colored immunofluorescence and flow cytometry was employed to demonstrate the binding of the IRTA5 HuMAbs to peripheral blood B cells. CD19 is a cell surface marker that can be used to distinguish peripheral blood B lymphocytes. Human peripheral blood mononuclear cells were incubated with biotinylated 2G5, biotinylated 7G8, or an isotype control biotinylated human Ab. Cells were washed and were incubated with FITC-labeled streptavidin together with a phycoerythrin-labeled anti-CD19 antibody. Cells were washed and analyzed by flow cytometry. The results are depicted in FIG. 10A. Lymphocytes were gated as black “dots” and monocytes were gated as grey “dots”. Wavelengths were selected to screen for FITC (FL1) and phycoerythrin (FL2) signaling. CD19+ B cells showed high level binding to the phycoerythrin-labeled anti-CD19 antibody (abscissa). 2G5+ or 7G8+ cells (ordinate) were also predominantly CD19+, localizing to the double-positive, upper right quadrant. These data demonstrate that the IRTA5 protein, as assessed by HuMAb 2G5 and 7G8 binding, is expressed by the majority, if not all, of normal peripheral blood B lymphocytes.

Two colored immunofluorescence and flow cytometry was also used to test for binding of the IRTA5 HuMAbs to peripheral blood T cells, dendritic cells, monocytes or natural killer (NK) cells. CD3, CD1A, CD14, and CD56 are cell surface marker that can be used to distinguish peripheral blood T lymphocytes, peripheral blood dendritic cells, peripheral blood monocytes, and peripheral blood NK cells, respectively. Biotinylated 2G5, 7G8, or isotype control antibody and phycoerythrin-labeled marker antibodies (CD3, CD1A, CD14, and CD56) were used in flow cytometry analysis, as described above. The results are depicted in FIG. 10B. Lymphocytes were gated as black “dots” and monocytes were gated as grey “dots”. Wavelengths were selected to screen for FITC (FL1) and phycoerythrin (FL2) signaling. The IRTA5 HuMAbs 2G5 and 7G8 did not bind to CD3+ peripheral blood T cells, CD1A+ peripheral blood dendritic cells, CD14+ peripheral blood monocytes, or CD56+ peripheral blood NK cells, confirming the B cell specific expression of IRTA5.

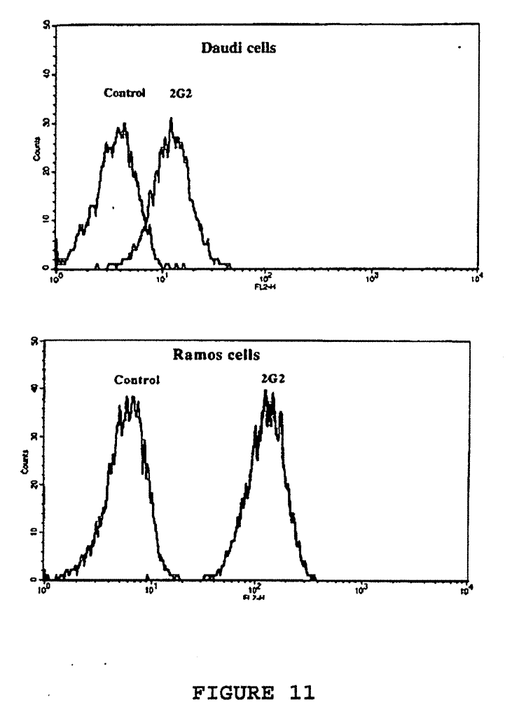

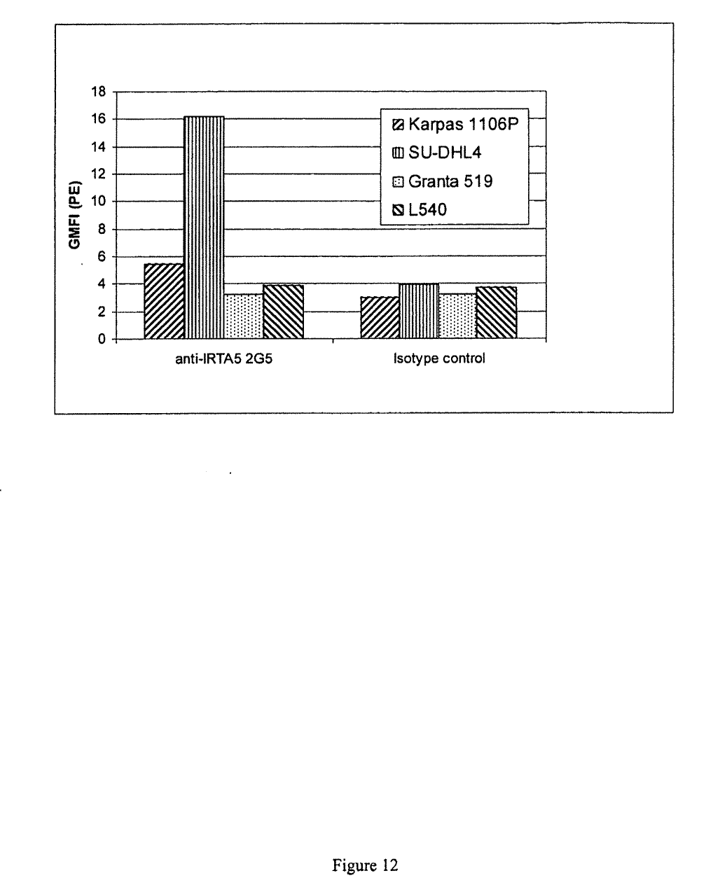

Binding of the IRTA5 HuMAbs to the B cell tumor lines Daudi (ATCC CCL-213), Ramos (ATCC CRL-1596), Karpas 1106P (DSMZ ACC 545), SU-DHL-4 (DSMZ ACC 495), Granta 519 (DSMZ ACC 342), and L-540 (DSMZ ACC 72) was assessed by flow cytometry. The cell lines were incubated with each of the IRTA5 HuMAbs or a control human antibody, washed and detected by a phycoerythrin-labeled anti-human secondary antibody. FIG. 11 represents a histogram which shows binding of the IRTA5 HuMAb 2G2 to Daudi and Ramos cells as compared to the control human antibody. The remaining 6 IRTA5 HuMAbs show a similar binding pattern (data not shown). These data show that the IRTA5 protein is expressed on the surface of the Daudi and Ramos tumor cell lines of B cell origin. FIG. 12 shows the binding of the IRTA5 HuMAb 2G5 to Karpas 1106P, SU-DHL-4, Granta 519, and L-540 cells as compared to an isotype control antibody. This data shows that the IRTA5 antibody has increased binding to the SU-DHL-4 B-cell tumor line, as measured by the mean fluorescent intensity (MFI) of staining. Together, these data demonstrate that certain B-cell tumor lines express the IRTA5 protein on the cell surface.

| IRTA-5 | |
|---|---|
| (SEQ ID NO: 37) | |
| 1 | mlprllllic aplcepaelf liaspshpte gspvtltckm pflqssdaqf qfcffrdtra |
| 61 | lgpgwssspk lqiaamwked tgsywceaqt maskvlrsrr sqinvhrvpv advsletqpp |
| 121 | ggqvmegdrl vlicsvamgt gditflwykg avglnlqskt qrsltaeyei psvresdaeq |
| 181 | yycvaengyg pspsglvsit vripvsrpil mlrapraqaa vedvlelhce alrgsppily |
| 241 | wfyheditlg srsapsggga sfnlslteeh sgnysceann glgaqrseav tlnftvptga |
| 301 | rsnhltsgvi egllstlgpa tvallfcygl krkigrrsar dplrslpspl pqeftylnsp |
| 361 | tpgqlqpiye nvnvvsgdev yslayynqpe qesvaaetlg thmedkvsld iysrlrkani |
| 421 | tdvdyedam |
| IRTA-1 | |
| (SEQ ID NO: 38) | |
| 1 | mllwasllaf apvcgqsaaa hkpvisvhpp wttffkgerv tltcngfqfy atekttwyhr |
| 61 | hywgekltlt pgntlevres glyrcqargs prsnpvrllf ssdslilqap ysvfegdtlv |
| 121 | lrchrrrkek ltavkytwng nilsisnksw dllipqassn nngnyrcigy gdendvfrsn |
| 181 | fkiikiqelf phpelkatds qptegnsvnl scetqlpper sdtplhfnff rdgevilsdw |
| 241 | stypelqlpt vwrensgsyw cgaetvrgni hkhspslqih vqripvsgvl letqpsggqa |
| 301 | vegemlvlvc svaegtgdtt fswhredmqe slgrktqrsl raelelpair qshaggyyct |
| 361 | adnsygpvqs mvlnvtvret pgnrdglvaa gatggllsal llavallfhc wrrrksgvgf |
| 421 | lgdetrlppa pgpgesshsi cpaqvelqsl yvdvhpkkgd lvyseiqttq lgeeeeants |
| 481 | rtlledkdvs vvysevktqh pdnsagkiss kdees |
| IRTA-2 | |
| (SEQ ID NO: 39) | |
| 1 | mllwvillvl apvsgqfart prpiiflqpp wttvfqgerv tltckgfrfy spqktkwyhr |
| 61 | ylgkeilret pdnilevqes geyrcqaqgs plsspvhldf ssaslilqap lsvfegdsvv |
| 121 | lrcrakaevt lnntiykndn vlaflnkrtd fhiphaclkd ngayrctgyk esccpvssnt |
| 181 | vkiqvqepft rpvlrassfq pisgnpvtlt cetqlslers dvplrfrffr ddqtlglgws |
| 241 | lspnfqitam wskdsgfywc kaatmphsvi sdsprswiqv qipashpvlt lspekalnfe |
| 301 | gtkvtlhcet qedslrtlyr fyhegvplrh ksvrcergas isfslttens gnyyctadng |
| 361 | lgakpskavs lsvtvpvshp vlnlsspedl ifegakvtlh ceaqrgslpi lygfhhedaa |
| 421 | lerrsansag gvaisfslta ehsgnyycta dngfgpqrsk avslsitvpv shpvltlssa |
| 481 | ealtfegatv tlhcevqrgs pqilyqfyhe dmplwssstp svgrvsfsfs lteghsgnyy |
| 541 | ctadngfgpq rsevvslfvt vpvsrpiltl rvpraqavvg dllelhceap rgsppilywf |
| 601 | yhedvtlgss sapsggeasf nlsltaehsg nysceanngl vaqhsdtisl svivpvsrpi |
| 661 | ltfrapraqa vvgdllelhc ealrgsspil ywfyhedvtl gkisapsggg asfnlsltte |
| 721 | hsgiyscead ngpeaqrsem vtlkvavpvs rpvltlrapg thaavgdlle lhcealrgsp |
| 781 | lilyrffhed vtlgnrssps ggaslnlslt aehsgnysce adnglgaqrs etvtlyitgl |
| 841 | tanrsgpfat gvaggllsia glaagallly cwlsrkagrk pasdparspp dsdsqeptyh |
| 901 | nvpaweelqp vytnanprge nvvysevrii qekkkhavas dprhlrnkgs piiysevkva |
| 961 | stpvsgslfl assaphr |
| IRTA-3 | |
| (SEQ ID NO: 40) | |
| 1 | mllwllllil tpgreqsgva pkavlllnpp wstafkgekv alicssishs laqgdtywyh |
| 61 | dekllkikhd kiqitepgny qcktrgssls davhvefspd wlilqalhpv fegdnvilrc |
| 121 | qgkdnknthq kvyykdgkql pnsynlekit vnsvsrdnsk yhctayrkfy ildievtskp |
| 181 | lniqvqelfl hpvlrassst piegspmtlt cetqlspqrp dvqlqfslfr dsqtlglgws |
| 241 | rsprlqipam wtedsgsywc evetvthsik krslrsqirv qrvpvsnvnl eirptggqli |
| 301 | egenmvlics vaqgsgtvtf swhkegrvrs lgrktqrsll aelhvltvke sdagryycaa |
| 361 | dnvhspilst wirvtvripv shpvltfrap rahtvvgdll elhceslrgs ppilyrfyhe |
| 421 | dvtlgnssap sgggasfnls ltaehsgnys cdadnglgaq hshgvslrvt vpvsrpvltl |
| 481 | rapgaqavvg dllelhcesl rgsfpilywf yheddtlgni sahsgggasf nlslttehsg |
| 541 | nysceadngl gaqhskvvtl nvtgtsrnrt gltaagitgl vlsilvlaaa aallhyarar |
| 601 | rkpgglsatg tsshspsecq epsssrpsri dpqepthskp lapmelepmy snvnpgdsnp |
| 661 | iysqiwsiqh tkensancpm mhqeheeltv lyselkkthp ddsageassr graheeddee |
| 721 | nyenvprvll asdh |
| IRTA-4 | |
| (SEQ ID NO: 41) | |
| 1 | mllwsllvif davteqadsl tlvapssvfe gdsivlkcqg eqnwkiqkma yhkdnkelsv |
| 61 | fkkfsdfliq savlsdsgny fcstkgqlfl wdktsnivki kvqelfqrpv ltassgqple |
| 121 | ggpvslkcet rlspqrldvq lqfcffrenq vlgsgwsssp elqisavwse dtgsywckae |
| 181 | tvthrirkqs lqsqihvqri pisnvsleir apggqvtegq klillcsvag gtgnvtfswy |
| 241 | reatgtsmgk ktqrslsael eipavkesda gkyycradng hvpiqskvvn ipvripvsrp |
| 301 | vltlrspgaq aavgdllelh cealrgsppi lyqfyhedvt lgnssapsgg gasfnlslta |
| 361 | ehsgnyscea nnglgaqcse avpvsisgpd gyrrdlmtag vlwglfgvlg ftgvalllya |
| 421 | lfhkisgess atneprgasr pnpqeftyss ptpdmeelqp vyvnvgsvdv dvvysqvwsm |
| 481 | qqpessanir tllenkdsqv iyssvkks |

SUMMARY OF SEQUENCE LISTING

| SEQ ID NO: | SEQUENCE |
|---|---|
| 1 | VH CDR1 a.a. 2G5 |
| 2 | VH CDR1 a.a. 5A2 |
| 3 | VH CDR1 a.a. 7G8 |
| 4 | VH CDR2 a.a. 2G5 |
| 5 | VH CDR2 a.a. 5A2 |
| 6 | VH CDR2 a.a. 7G8 |
| 7 | VH CDR3 a.a. 2G5 |
| 8 | VH CDR3 a.a. 5A2 |
| 9 | VH CDR3 a.a. 7G8 |
| 10 | VK CDR1 a.a. 2G5 |
| 11 | VK CDR1 a.a. 5A2 |
| 12 | VK CDR1 a.a. 7G8 |
| 13 | VK CDR2 a.a. 2G5 |
| 14 | VK CDR2 a.a. 5A2 |
| 15 | VK CDR2 a.a. 7G8 |
| 16 | VK CDR3 a.a. 2G5 |
| 17 | VK CDR3 a.a. 5A2 |
| 18 | VK CDR3 a.a. 7G8 |
| 19 | VH a.a. 2G5 |
| 20 | VH a.a. 5A2 |
| 21 | VH a.a. 7G8 |
| 22 | VK a.a. 2G5 |
| 23 | VK a.a. 5A2 |
| 24 | VK a.a. 7G8 |
| 25 | VH n.t. 2G5 |
| 26 | VH n.t. 5A2 |
| 27 | VH n.t. 7G8 |
| 28 | VK n.t. 2G5 |
| 29 | VK n.t. 5A2 |
| 30 | VK n.t. 7G8 |
| 31 | VH 3-33 germline a.a. |
| 32 | VH DP44 germline a.a. |
| 33 | VK L6 germline a.a. |
| 34 | VH 3-23 germline a.a. |
| 35 | VH 3-7 germline a.a. |
| 36 | VH 7G8(mut) a.a. |
| 37 | IRTA-5 a.a. |
| 38 | IRTA-1 a.a. |
| 39 | IRTA-2 a.a. |
| 40 | IRTA-3 a.a. |
| 41 | IRTA-4 a.a. |

We claim:

1. An isolated monoclonal antibody, or an antigen-binding portion thereof, wherein the antibody:
(a) binds to human Immune Receptor Translocation Associated-5 (IRTA-5) amino acid sequence of SEQ ID NO: 37 with a K_{D} of 5×10⁻⁸ M or less;
(b) selectively binds to human IRTA-5 having an amino acid sequence of SEQ ID NO: 37 by greater than about 10:1 as measured by immunoassay over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO: 41; and
(c) binds to human B lymphocytes and to B cell tumor lines but does not bind to CD3⁺ peripheral blood T cells, CD1A⁺ peripheral blood dendritic cells, CD14⁺ peripheral blood monocytes, or CD56⁺ peripheral blood natural killer cells.

2. The antibody of claim 1, which is a human antibody.

3. The antibody of claim 2, which is a full-length antibody of an IgG1 or IgG4 isotype.

4. The antibody of claim 2, which is an antibody fragment or a single chain antibody.

5. The antibody of claim 2, wherein said antibody binds to human IRTA-5 with a K_{D} of 3×10⁻⁸ M or less.

6. The antibody of claim 2, wherein said antibody binds to human IRTA-5 with a K_{D} of 1×10⁻⁹ M or less.

7. The antibody of claim 2, wherein said antibody binds to human IRTA-5 with a K_{D} of 0.1×10⁻⁹ M or less.

8. The antibody of claim 2, wherein said antibody binds to human IRTA-5 with a K_{D} of 0.05×10⁻⁹ M or less.

9. The antibody of claim 2, wherein said antibody binds to human IRTA-5 with a K_{D} of between 1×10⁻⁹ and 1×10⁻¹¹ M.

10. The antibody of claim 1, wherein the B cell tumor lines are selected from the group consisting of Daudi, Ramos, and SU-DHL-4 cell lines.

11. An isolated monoclonal antibody, or antigen binding portion thereof, wherein the antibody cross-competes for binding to IRTA-5 with a reference antibody, wherein the reference antibody:
(a) binds to human IRTA-5 amino acid sequence of SEQ ID NO: 37 with a K_{D} of 5×10⁻⁸ M or less;
(b) selectively binds to human IRTA-5 having an amino acid sequence of SEQ ID NO: 37 by greater than about 10:1 as measured by immunoassay over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO:41; and
(c) binds to human B lymphocytes and to B cell tumor lines but does not bind to CD3⁺ peripheral blood T cells, CD1A⁺ peripheral blood dendritic cells, CD14⁺ peripheral blood monocytes, or CD56⁺ peripheral blood natural killer cells.

12. The antibody of claim 11, wherein the reference antibody comprises:
(a) a heavy chain variable region comprising the amino acid sequence of SEQ ID NO: 19; and
(b) a light chain variable region comprising the amino acid sequence of SEQ ID NO: 22.

13. The antibody of claim 11, wherein the reference antibody comprises:
(a) a heavy chain variable region comprising the amino acid sequence of SEQ ID NO: 20; and
(b) a light chain variable region comprising the amino acid sequence of SEQ ID NO: 23.

14. The antibody of claim 11, wherein the reference antibody comprises:
(a) a heavy chain variable region comprising the amino acid sequence of SEQ ID NO: 21; and
(b) a light chain variable region comprising the amino acid sequence of SEQ ID NO: 24.

15. An isolated monoclonal antibody, or an antigen-binding portion thereof, comprising a heavy chain variable region that is the product of a human V_{H} 3-33 gene, a human V_{H} DP44 gene, a human V_{H} 3-23 gene or a human V_{H} 3-7 gene, wherein the antibody specifically binds IRTA-5 amino acid sequence of SEQ ID NO: 37.

16. An isolated monoclonal antibody, or an antigen-binding portion thereof, comprising a light chain variable region that is the product of a human V_{K} L6 gene, wherein the antibody specifically binds IRTA-5 amino acid sequence of SEQ ID NO: 37.

17. The isolated monoclonal antibody, or an antigen-binding portion thereof, of claim 16 further comprising:
a heavy chain variable region of a human V_{H} 3-33, V_{H} DP44, V_{H} 3-23, or V_{H} 3-7 gene.

18. The antibody of claim 17, which comprises a heavy chain variable region of a human V_{H} 3-33 gene and a light chain variable region of a human V_{K} L6 gene.

19. The antibody of claim 17, which comprises a heavy chain variable region of a human V_{H} DP44 gene and a light chain variable region of a human V_{K} L6 gene.

20. The antibody of claim 17, which comprises a heavy chain variable region of a human V_{H} 3-23 gene and a light chain variable region of a human V_{K} L6 gene.

21. The antibody of claim 17, which comprises a heavy chain variable region of a human V_{H} 3-7 gene and a light chain variable region of a human V_{K} L6 gene.

22. The antibody of claim 17, wherein the antibody selectively binds to human IRTA-5 having an amino acid sequence of SEQ ID NO: 37 by greater than about 10:1 as measured by immunoassay over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO: 41.

23. The antibody of claim 1, which comprises:
(a) a heavy chain variable region CDR1 comprising SEQ ID NO: 1;
(b) a heavy chain variable region CDR2 comprising SEQ ID NO: 4;
(c) a heavy chain variable region CDR3 comprising SEQ ID NO: 7;
(d) a light chain variable region CDR1 comprising SEQ ID NO: 10;
(e) a light chain variable region CDR2 comprising SEQ ID NO: 13; and
(f) a light chain variable region CDR3 comprising SEQ ID NO: 16.

24. The antibody of claim 1, which comprises:
(a) a heavy chain variable region CDR1 comprising SEQ ID NO: 2;
(b) a heavy chain variable region CDR2 comprising SEQ ID NO: 5;
(c) a heavy chain variable region CDR3 comprising SEQ ID NO: 8:
(d) a light chain variable region CDR1 comprising SEQ ID NO: 11;
(e) a light chain variable region CDR2 comprising SEQ ID NO: 14;
(f) a light chain variable region CDR3 comprising SEQ ID NO: 17.

25. The antibody of claim 1, which comprises:
(a) a heavy chain variable region CDR1 comprising SEQ ID NO: 3;
(b) a heavy chain variable region CDR2 comprising SEQ ID NO: 6;
(c) a heavy chain variable region CDR3 comprising SEQ ID NO: 9;
(d) a light chain variable region CDR1 comprising SEQ ID NO: 12;
(e) a light chain variable region CDR2 comprising SEQ ID NO: 15; and
(f) a light chain variable region CDR3 comprising SEQ ID NO: 18.

26. The antibody of claim 1, which comprises:
(a) a heavy chain variable region comprising the amino acid sequence of SEQ ID NO: 19; and
(b) a light chain variable region comprising the amino acid sequence of SEQ ID NO: 22.

27. The antibody of claim 1, which comprises:
(a) a heavy chain variable region comprising the amino acid sequence of SEQ ID NO: 20; and
(b) a light chain variable region comprising the amino acid sequence of SEQ ID NO: 23.

28. The antibody of claim 1, which comprises:
(a) a heavy chain variable region comprising the amino acid sequence of SEQ ID NO: 21 or 36; and
(b) a light chain variable region comprising the amino acid sequence of SEQ ID NO: 24.

29. A composition comprising the antibody, or antigen-binding portion thereof, of claim 1, and a pharmaceutically acceptable carrier.

30. The antibody of claim 1, wherein the antibody selectively binds to human IRTA-5 having an amino acid sequence of SEQ ID NO: 37 by greater than about 100:1 over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO: 41.

31. The antibody of claim 11, wherein the antibody selectively binds to human IRTA-5 having an amino acid sequence of SEQ ID NO: 37 by greater than about 100:1 over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO: 41.

32. The antibody of claim 22, wherein the antibody selectively binds to human IRTA-5 having an amino acid sequence of SEQ ID NO: 37 by greater than about 100:1 over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO: 41.

33. An isolated monoclonal antibody, or an antigen-binding portion thereof, wherein the antibody:
(a) binds to human Immune Receptor Translocation Associated-5 (IRTA-5) having an amino acid sequence of SEQ ID NO: 37 with a K_{D} of 5×10⁻⁸ M or less; and
(b) selectively binds to human IRTA-5 having an amino acid sequence of SEQ ID NO: 37 by greater than about 10:1 as measured by immunoassay over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO:41.

34. The antibody of claim 33, wherein the antibody selectively binds to human IRTA-5 having an amino acid sequence of SEQ ID NO: 37 by greater than about 100:1 over human IRTA-1 having an amino acid sequence of SEQ ID NO: 38, IRTA-2 having an amino acid sequence of SEQ ID NO: 39, IRTA-3 having an amino acid sequence of SEQ ID NO: 40, and/or IRTA-4 having an amino acid sequence of SEQ ID NO: 41.
